(19) United States
(12) Patent Application Publication
(10) Pub. No.: US 2018/0204643 A1
(43) Pub. Date: Jul. 19, 2018

(54) Method and Apparatus for Generating Medical Data Transmitted and Received Between Equipments Related to Medical Imaging

(71) Applicant: IRM INC., Gyeonggi-do (KR)

(72) Inventor: Seung Wook Choi, Gyeonggi-do (KR)

(21) Appl. No.: 15/740,422

(22) PCT Filed: Jul. 21, 2016
(86) PCT No.: PCT/KR2016/007931

§ 371 Date: Dec. 28, 2017

(30) Foreign Application Priority Data
Jul. 21, 2015 (KR) 10-2015-0103010
Jul. 20, 2016 (KR) 10-2016-0091798

(51) Int. Cl. G16H 50/70; G16H 10/60; G06F 17/30

(52) U.S. Cl. CPC G16H 50/70; G06F 17/30595; G16H 10/60

(57) ABSTRACT

A method of generating medical data includes: determining a manipulation type based on an order of generating at least one medical image; selecting a first subject and a second subject from among a plurality of medical systems, based on the determined manipulation type; and generating link data for linking first medical data generated by the first subject with second medical data generated by the second subject.

### TECHNICAL FIELD

The present inventive concept relates to a method and an apparatus for generating medical data exchanged between devices related to medical imaging.

### BACKGROUND ART

As medical technologies and communication technologies have developed, in recent times, attempts have actively been made to integrate medical industries and communication industries. In particular, research has been conducted into technologies for generating desired types of medical information by processing a diagnosis result of an subject (for example, a patient) and exchanging the desired types of medical information between devices located apart from each other.

### DETAILED DESCRIPTION OF THE DISCLOSURE

### Technical Problem

Embodiments disclosed herein relate to a method and an apparatus for generating medical data exchanged between devices related to medical imaging. Embodiments disclosed herein relate to a computer-readable recording medium having embodied thereon a program for executing the method. Embodiments disclosed herein are not limited thereto, and may relate to other technical problems.

### Technical Solution

Provided is a method of generating medical data, the method including: determining a manipulation type based on an order of generating at least one medical image; selecting a first subject and a second subject from among a plurality of medical systems, based on the determined manipulation type; and generating link data for linking first medical data generated by the first subject with second medical data generated by the second subject.

The determining the manipulation type may include determining the manipulation type based on whether or not the order exists and based on a number of the order.

The determining the manipulation type may include determining any one of a first manipulation type for executing a single examination in response to a single order, a second manipulation type for executing a single examination without the order, a third manipulation type for executing a plurality of examinations in response to a single order, and a fourth manipulation type for executing a single examination in response to a plurality of orders.

The order of generating at least one medical image may include an order of generating at least one medical image indicating a region of interest included in a subject to be examined.

The selecting the first subject and the second subject may include selecting the first subject and the second subject from among an electronic medical record system, a medical image generating modality, a picture archiving communication system, a radiology information system, and a laboratory information system.

Provided is a computer-readable recording medium having embodied thereon a program for executing the method described above.

Provided is an apparatus for generating medical data, the apparatus including: a determination portion configured to determine a manipulation type based on an order of generating at least one medical image; a selection portion configured to select a first subject and a second subject from among a plurality of medical systems, based on the determined manipulation type; and a generation portion configured to generate link data for linking first medical data generated by the first subject with second medical data generated by the second subject.

The determination portion may be configured to determine the manipulation type based on whether or not the order exists and based on a number of the order.

The determination portion may be configured to determine any one of a first manipulation type for executing a single examination in response to a single order, a second manipulation type for executing a single examination without the order, a third manipulation type for executing a plurality of examinations in response to a single order, and a fourth manipulation type for executing a single examination in response to a plurality of orders.

The order may include an order of generating at least one medical image indicating a region of interest included in a subject to be examined.

The selection portion may be configured to select the first subject and the second subject from among an electronic medical record system, a medical image generating modality, a picture archiving communication system, a radiology information system, and a laboratory information system.

### Advantageous Effects of the Disclosure

Medical data related to medical imaging may be automatically generated without intervention of a user. In detail, with respect to an order of capturing a medical image, a captured medical image, and a medical operation, link data for linking data generated by each of medical devices may be automatically generated without intervention of the user.

Also, based on the generation or manipulation of the link data for linking the medical data, it is possible to link study data, series data, and/or instance data based on digital imaging and communication in medicine (DICOM), a standard in the field of medicine, to general document data and/or document set data that are not based on the standard in the field of medicine. Thus, regardless of whether data complies with the standard of the field of medicine, medical data may be managed in an integrated manner. Also, a system, storage, etc. configured to integrate and manage the medical data may be implemented.

### DESCRIPTION OF THE DRAWINGS

FIG. 1 is a diagram of an example of a medical system according to an embodiment.

FIG. 2 is a flowchart of an example of a method of generating medical data, according to an embodiment.

FIG. 3 is a diagram for describing an example in which an apparatus for generating medical data generates link data.

FIGS. 4A through 7B are diagrams for describing examples in which an apparatus for generating medical data, according to an embodiment, generates link data based on a first manipulation type.

FIGS. 8A through 9B are diagrams for describing examples in which an apparatus for generating medical data, according to an embodiment, generates link data based on a second manipulation type.

FIGS. 10A through 14D are diagrams for describing examples in which an apparatus for generating medical data, according to an embodiment, generates link data based on a fourth manipulation type.

FIG. 15 is a view of an example of a screen on which medical data is output, according to an embodiment.

### BEST MODE

According to an aspect of the present inventive concept, there is provided a method of generating medical data, the method including: determining a manipulation type based on an order of generating at least one medical image; selecting a first subject and a second subject from among a plurality of medical systems, based on the determined manipulation type; and generating link data for linking first medical data generated by the first subject with second medical data generated by the second subject.

### MODE OF THE DISCLOSURE

The terms used in the present disclosure are selected from among common terms that are currently widely used in consideration of their function in the present disclosure. However, the terms may be different according to an intention of one of ordinary skill in the art, a precedent, or the advent of new technology. Also, in particular cases, the terms are discretionally selected by the applicant of the present disclosure, and the meaning of those terms will be described in detail in the corresponding part of the detailed description. Therefore, the terms used in the present disclosure are not merely designations of the terms, but the terms are defined based on the meaning of the terms and content throughout the present disclosure.

Throughout the present application, when a part “includes” an element, it is to be understood that the part additionally includes other elements rather than excluding other elements as long as there is no particular opposing recitation. Also, the terms such as “ . . . unit,” “module,” or the like used in the present application indicate an unit, which processes at least one function or motion, and the unit may be implemented by hardware or software, or by a combination of hardware and software.

The present disclosure will now be described more fully with reference to the accompanying drawings for those of ordinary skill in the art to be able to perform the present disclosure without any difficulty. The disclosure may, however, be embodied in many different forms and should not be construed as being limited to the embodiments set forth herein.

Hereinafter, embodiments of the present disclosure will be described in detail by referring to the accompanying drawings.

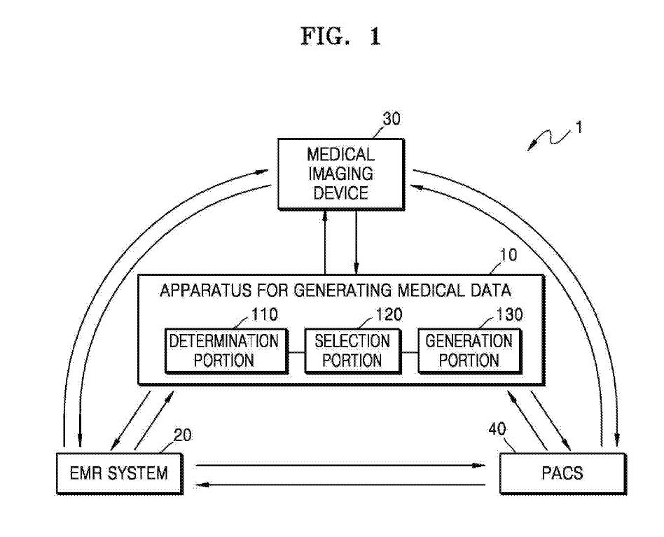

FIG. 1 is a diagram of an example of a medical system 1 according to an embodiment.

Referring to FIG. 1, the medical system 1 may include an apparatus for generating medical data 10, an electronic medical record system 20, a medical imaging device 30, and a picture archiving communication system 40. Also, although it is not illustrated in FIG. 1, the medical system 1 may include a radiology information system and/or a laboratory information system.

Each of the electronic medical record system 20, the medical imaging device 30, and the picture archiving communication system 40 included in the medical system 1 may generate medical data and transmit and receive the generated medical data to and from each another. Here, the medical data may refer to any one of text, image, video, waveforms, and general documents. For example, the electronic medical record system 20, the medical imaging device 30, and the picture archiving communication system 40 may generate the medical data including an order of generating a medical image, the medical image, information notifying the generation of the medical image, etc., and transmit and receive the generated medical data to and from each another.

The apparatus for generating medical data 10 may select a first subject and a second subject from among the devices (that is, the electronic medical record system 20, the medical imaging device 30, the picture archiving communication system 40, and the radiology information system and/or the laboratory information system) included in the medical system 1, and may generate link data for linking first medical data generated by the first subject with second medical data generated by the second subject. Here, the first subject and the second subject may be selected based on a manipulation type determined based on the order.

Meanwhile, the first subject and the second subject selected by the apparatus for generating medical data 10 are not limited to the electronic medical record system 20, the medical imaging device 30, the picture archiving communication system 40, the radiology information system, and the laboratory information system. In other words, the medical system 1 may include other devices in addition to the described systems and devices, and the apparatus for generating medical data 10 may select the first subject and the second subject from among the devices included in the medical system 1.

For example, the apparatus for generating medical data 10 may generate the link data for linking the first medical data generated by a transmitting device (that is, the electronic medical record system 20, the medical imaging device 30, the picture archiving communication system 40, the radiology information system, or the laboratory information system) with the second medical data generated by a receiving device (that is, the electronic medical record system 20, the medical imaging device 30, the picture archiving communication system 40, the radiology information system, or the laboratory information system). For example, when it is assumed that the electronic medical record system 20 transmits, to the medical imaging device 30, an order of capturing a medical image, the apparatus for generating medical data 10 may generate the link data for linking the first medical data generated by the electronic medical record system 20 with the second medical data generated by the medical imaging device 30. Thus, the order of the electronic medical record system 20 may be transmitted to the medical imaging device 30.

The electronic medical record system 20 refers to a system in which information (that is, medical record) related to treatment of a patient is computerized, input, managed, and stored. Here, the medical record refers to all information related to the patient, such as personal information, a medical history, a health state, a record of medical examination, a record of hospitalization or discharge from the hospital of the patient, etc.

The medical imaging device 30 refers to a modality configured to capture a medical image indicating a physical region of interest (ROI) of a patient. For example, the medical imaging device 30 may include an X-ray imaging device, a computerized tomography (CT) imaging device, an magnetic resonance imaging (MRI) device, a positron emission tomography (PET) imaging device, an ornithine carbamoyltransferase (OCT) imaging device, etc.

The picture archiving communication system 40 refers to a system to store or transmit a medical image. That is, the picture archiving communication system 40 refers to the system which is used by a user (for example, a medical expert, such as a doctor, etc.) to read the stored medical image and perform an image diagnosis.

The radiology information system refers to an analysis and management system of a medical image, such as an X-ray image, a CT image, an MRI image, etc. The radiology information system refers to a system configured to perform general managing functions of a department of radiology, such as management of an analysis report, management of reservations of capturing, management of reference sources for the image analysis, etc.

The laboratory information system refers to a network established among laboratories and treatment departments of a hospital. For example, through the laboratory information system, examination results of the laboratories are automatically input to examination items in a patient registration system so that a doctor in charge may identify the examination results via a terminal. In most hospitals, necessary examination items are indicated and delivered based on an order of a doctor, and thus, in many cases, a department of clinical pathology, a department of diagnostic radiology, etc., which do not directly contact a patient, may not know problems of the patient. Thus, a user may check a record of the patient via a terminal in each laboratory and review the sources necessary for the diagnosis.

The medical data generated by the devices (the apparatus for generating medical data 10, the electronic medical record system 20, the medical imaging device 30, and the picture archiving communication system 40) included in the medical system 1 may be linked to one another based on wired/wireless communication protocols. For example, the medical system 1 may provide various services, such as medical data generating, searching, storing, transferring, inquiring, exchanging, linking, etc., based on medical information standards, such as integrating the healthcare enterprise (IHE), digital imaging and communication in medicine (DICOM), and health level-7 (HL7), and international standards, such as the world wide web consortium (W3C) and the international organization for standardization (ISO).

The apparatus for generating medical data 10 may include a determination portion 110, a selection portion 120, and a generation portion 130. FIG. 1 illustrates that the determination portion 110, the selection portion 120, and the generation portion 130 are included in the apparatus for generating medical data 10 as separate modules. However, embodiments of the present disclosure are not limited thereto. That is, at least two of the determination portion 110, the selection portion 120, and the generation portion 130 may be implemented as a single module.

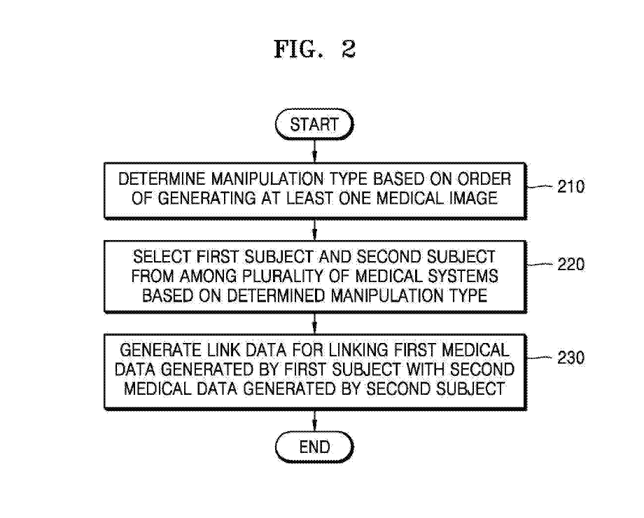

Hereinafter, examples of an operation of the apparatus for generating medical data 10 will be described by referring to FIGS. 2 through 14. Meanwhile, terms to be used in the description are defined as below. Also, hereinafter, the medical data according to the present disclosure is described as documents. In other words, the medical data may include all types of documents which may be processed by a general computing device, such as image, waveforms, measured values, etc.

One document may be included in a document set (hereinafter, DocSet) that is equal to or greater than 0. In other words, the document may not be included in the DocSet, or may be included in one or more DocSets. Also, a specific document has to be necessarily included in one group. Here, the group may refer to an actual organizational group, such as a hospital, a department, etc., or a virtual user group. Detailed aspects with respect to the group will be described later. The apparatus for generating medical data 10 may assign identification (ID) to each document. Types of IDs of the documents and definitions thereof according to an embodiment are shown as Table 1 below.

TABLE 1

| Types of IDs | Definitions |
|---|---|
| Document UID | Unique ID of document. Unique in specific |
| | group in which document is generated. |
| Document UUID | Unique ID of document, which is different from |
| | document UID. Unique in entire documents. For |
| | example, while document UID of first document |
| | is one, document UUID may vary whenever |
| | first document is transmitted (or processed). |
| Filter UID | ID indicating filter via which document is |
| | generated. |
| Patient ID | ID indicating patient related to document |

Each document has unique ID (hereinafter, UID) in the apparatus for generating medical data 10 according to an embodiment, wherein the uniqueness of the UID is guaranteed only in a specific group. Also, each document has unique UID (UUID), which is different from the UID, wherein the uniqueness of the UUID is guaranteed in the entire documents. The document according to an embodiment may correspond to a document entry in the IHE extended data service (XDS).

The DocSet denotes a virtual set of actual documents. All the processing in the apparatus for generating medical data 10 is executed in a unit of the DocSet. Also, a specific DocSet has to be necessarily included in one group. ID is given to each DocSet to identify the DocSet in the apparatus for generating medical data 10. Types of IDs related to the DocSet and definitions thereof according to an embodiment are shown as Table 2 below.

TABLE 2

| Types of IDs | Definitions |
|---|---|
| DocSet UID | Unique ID of DocSet. Unique in specific |
| | group in which DocSet is generated. |
| DocSet UUID | Unique ID of DocSet, which is different from |
| | DocSet UID. Unique in entire DocSets. For |
| | example, while DocSet UID of first DocSet |
| | is one, DocSet UUID may vary whenever |
| | first DocSet is transmitted (or processed). |
| DocSet Class UID | Indicating types of DocSet and used to |
| | designate input and output format of filter |
| DocSet Group UID | Used to indicate relations among DocSets |
| Filter UID | ID indicating filter via which document is |
| | generated. |
| Patient Id | ID indicating patient related to document |

The DocSet of the apparatus for generating medical data 10 according to an embodiment corresponds to a submission set in the IHE XDS.

Also, the DocSet necessarily has one DocSet Class. Here, the DocSet Class denotes a class of the DocSet. The DocSet Class is used to designate formats of an input and an output of a filter, and is indicated as DocSet Class UID in the apparatus for generating medical data 10.

FIG. 2 is a flowchart of an example of a method of generating medical data, according to an embodiment.

Referring to FIG. 2, the method of generating the medical data may include operations that are time-sequentially performed by the apparatus for generating medical data 10 illustrated in FIG. 1. Thus, even if omitted hereinafter, the aspects described above with reference to FIG. 1 should be understood to be applied to the method of generating the medical data of FIG. 2.

In operation S210, the determination portion 110 may determine a manipulation type based on an order of generating at least one medical image. Here, the order may include an order of generating, via a user, at least one medical image indicating a region of interest (ROI) of a subject to be examined (that is, a patient). Here, the ROI refers to an internal organ or a body part of the subject to be examined (that is, the patient), which is to be diagnosed by the user via the medical image.

Meanwhile, hereinafter, an “examination” denotes a set of a plurality of medical images indicating an ROI of a subject to be examined. For example, the examination may denote a set of a plurality of medical images in which a breast of a patient is captured. Thus, hereinafter, “to execute an examination” denotes to generate a plurality of medical images indicating an ROI of the subject to be examined.

Also, hereinafter, a “series” denotes a specific set of the plurality of medical images included in the examination, wherein the specific set is generated by classifying the plurality of medical images based on a predetermined reference. For example, when it is assumed that the examination denotes a set of a plurality of medical images in which a breast of a patient is captured, the series may denote a specific set of the plurality of images in which the breast of the patient is captured, wherein the specific set includes the images in which the breast of the patient is captured at the front. Also, hereinafter, the “image” denotes each of the plurality of medical images included in the examination or the series. Thus, hereinafter, the examination, the series, and the image have an inclusion relationship of “the examination ⊃ the series ⊃ the image.”

The determination portion 110 may determine a manipulation type based on whether or not an order exists and based on a number of the order. For example, the determination portion 110 may determine any one of a first manipulation type for executing a single examination in response to a single order, a second manipulation type for executing a single examination without the order, a third manipulation type for executing a plurality of examinations in response to a single order, and a fourth manipulation type for executing a single examination in response to a plurality of orders.

For example, the first manipulation type is a manipulation type whereby an examination indicating a breast of a subject to be examined is executed once, in response to an order of generating a medical image indicating the breast of the subject to be examined, and the third manipulation type is a manipulation type whereby the examination indicating the breast of the subject to be examined is executed in a plurality of times, in response to the order of generating the medical image indicating the breast of the subject to be examined. Also, the fourth manipulation type is a manipulation type whereby an examination simultaneously indicating the breast and a stomach of the subject to be examined is executed once, in response to an order of generating a medical image indicating the breast of the subject to be examined and an order of generating a medical image indicating the stomach of the subject to be examined.

For example, based on the IHE medical information standard, the first manipulation type may correspond to a simple case, the second manipulation type may correspond to an unscheduled case, the third manipulation type may correspond to an appended case, and the fourth manipulation type may correspond to a group case.

The selection portion 120 may select a first subject and a second subject based on the determined manipulation type. Here, the first subject refers to a device to transmit medical data, from among the electronic medical record system 20, the medical imaging device 30, and the picture archiving communication system 40 included in the medical system 1. Also, the second subject refers to a device to receive the medical data, from among the electronic medical record system 20, the medical imaging device 30, and the picture archiving communication system 40 included in the medical system 1.

Here, the transmitting medical data may include linking first medical data generated by the first subject with second medical data generated by the second subject, so that information included in the first medical data is included in the second medical data. Since the first subject and the second subject are different devices, the first medical data may not be executed by the second device. Thus, the apparatus for generating medical data 10 may generate link data for linking the first medical data generated by the first subject with the second medical data generated by the second subject, so that the information included in the first medical data may be included in the second medical data. Thus, the effects may be generated that certain information may be transmitted from the first subject to the second subject.

The generation portion 130 may generate the link data for linking the first medical data generated by the first subject with the second medical data generated by the second subject. Here, the generation portion 130 may generate the link data based on the manipulation type determined by the determination portion 110. The generating the link data according to an embodiment may include generating the link data for linking previous medical data with new medical data (that is, medical data newly generated by any one of the electronic medical record system 20, the medical imaging device 30, the picture archiving communication system 40, the radiology information system, and the laboratory information system). For example, the generation portion 130 may generate the link data for linking the previous medical data to a document and/or a DocSet according to an embodiment.

The link data denotes data for linking the first medical data with the second medical data based on communication protocols. Here, the communication protocols refer to communication protocols based on the medical information standards, such as IHE, DICOM, and HL7, and the international standards, such as the W3C and the ISO.

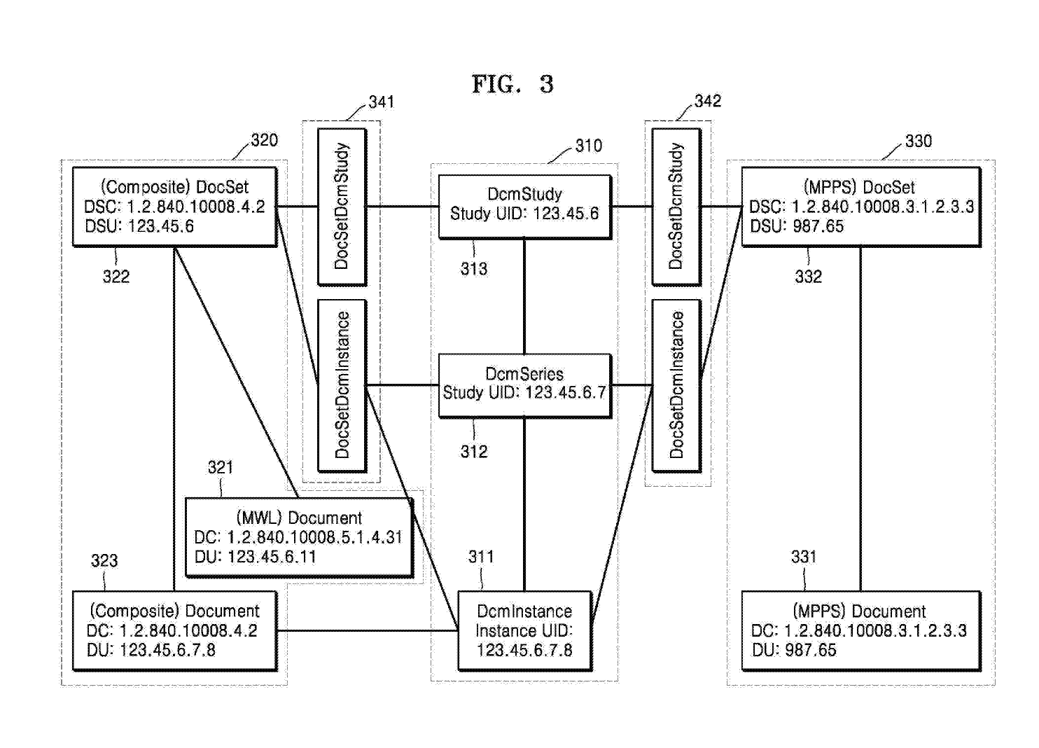

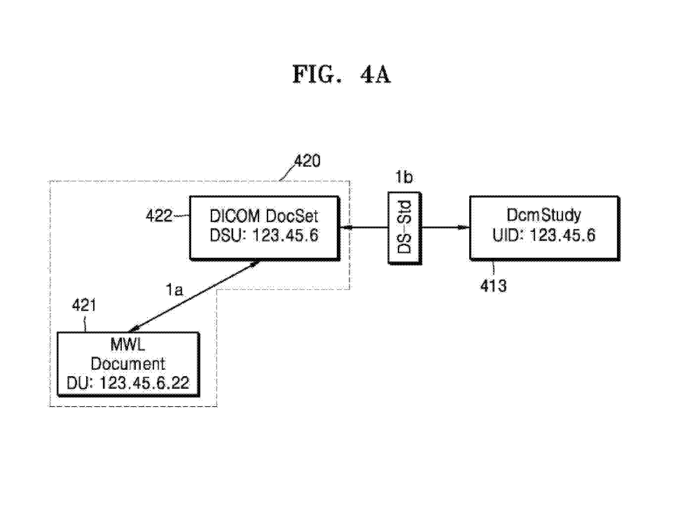

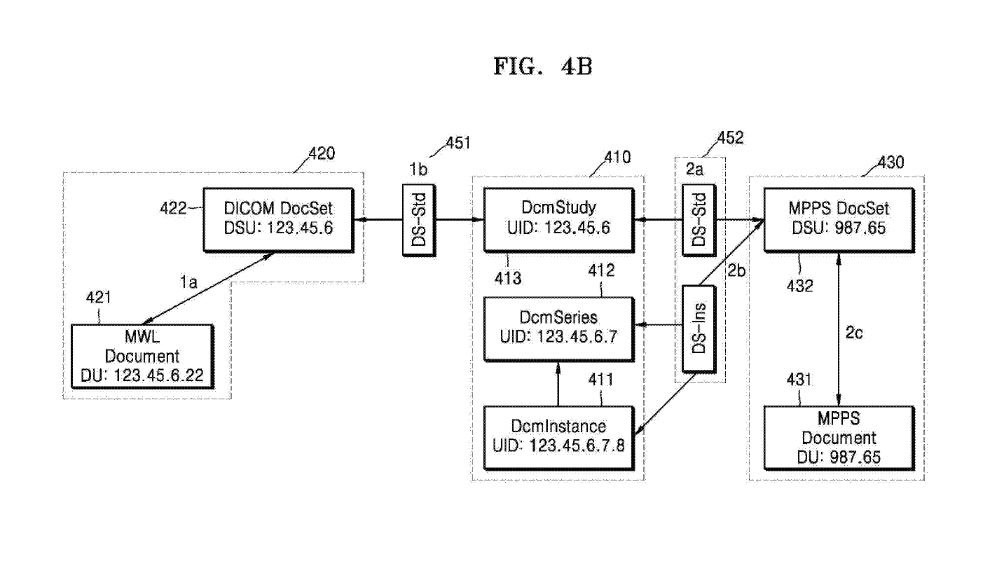

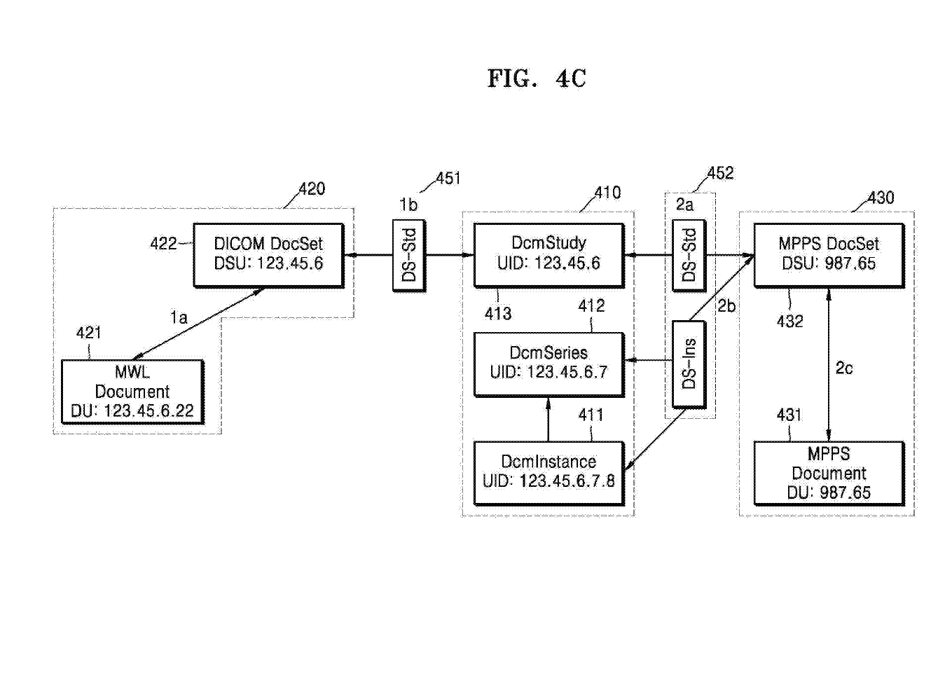

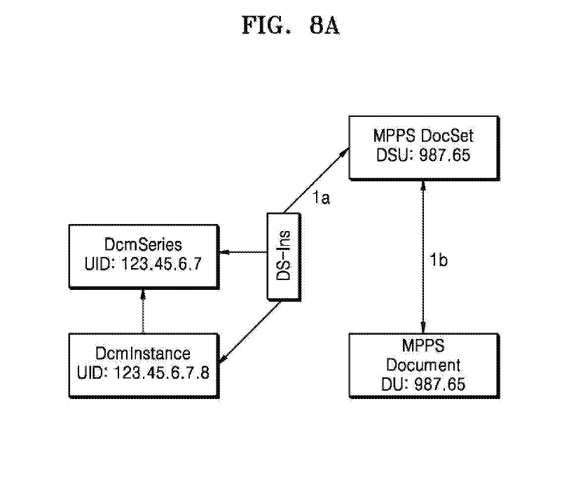

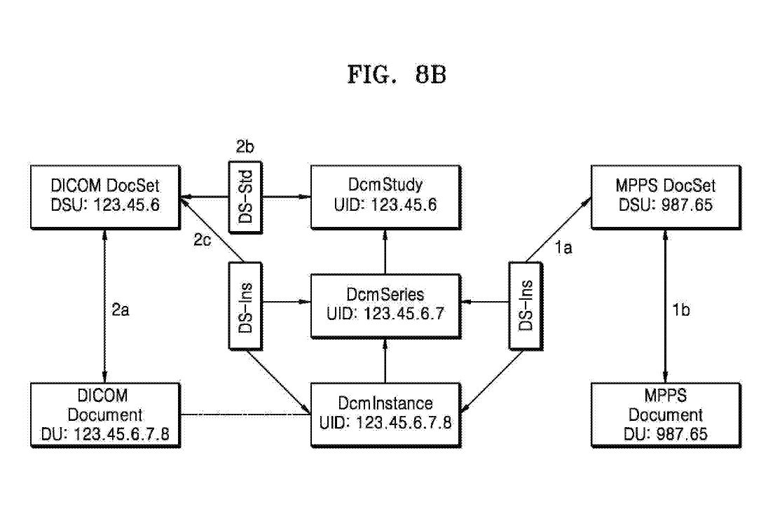

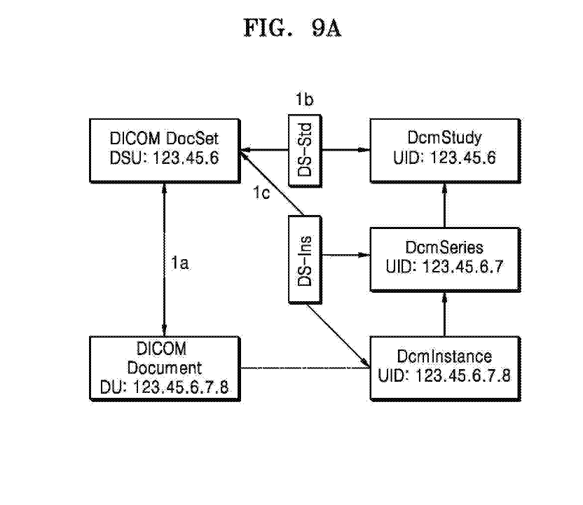

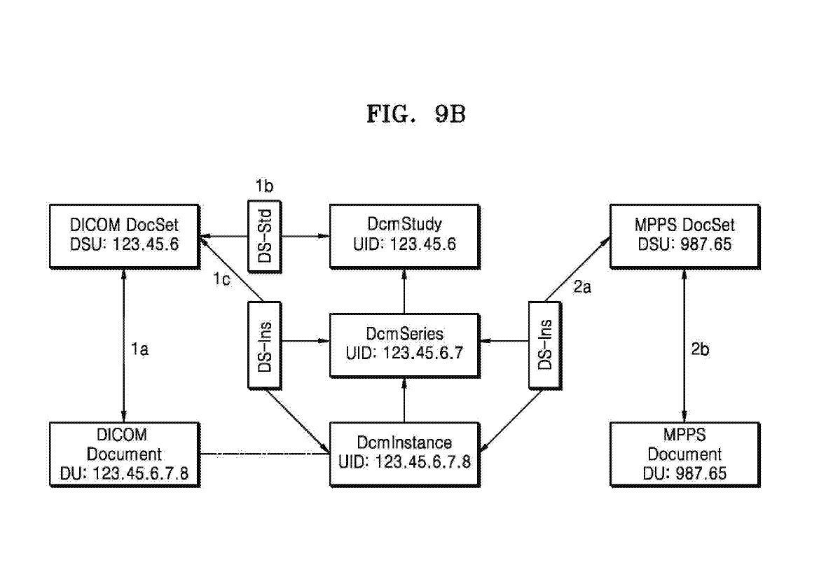

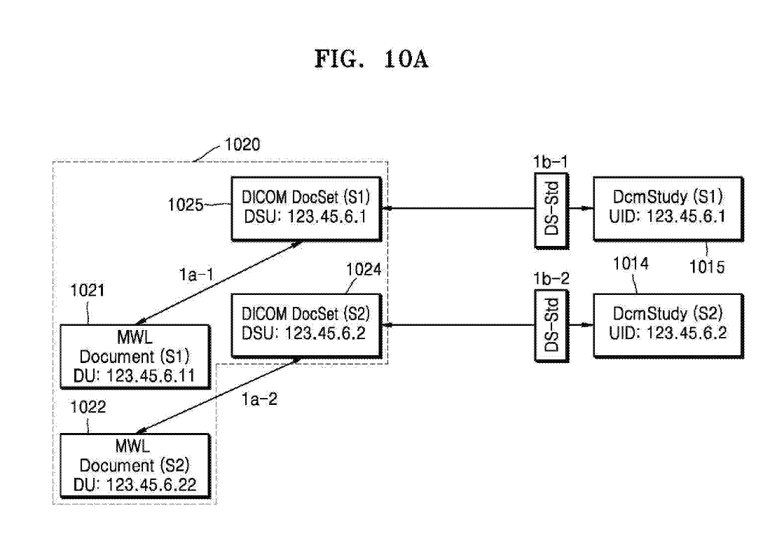

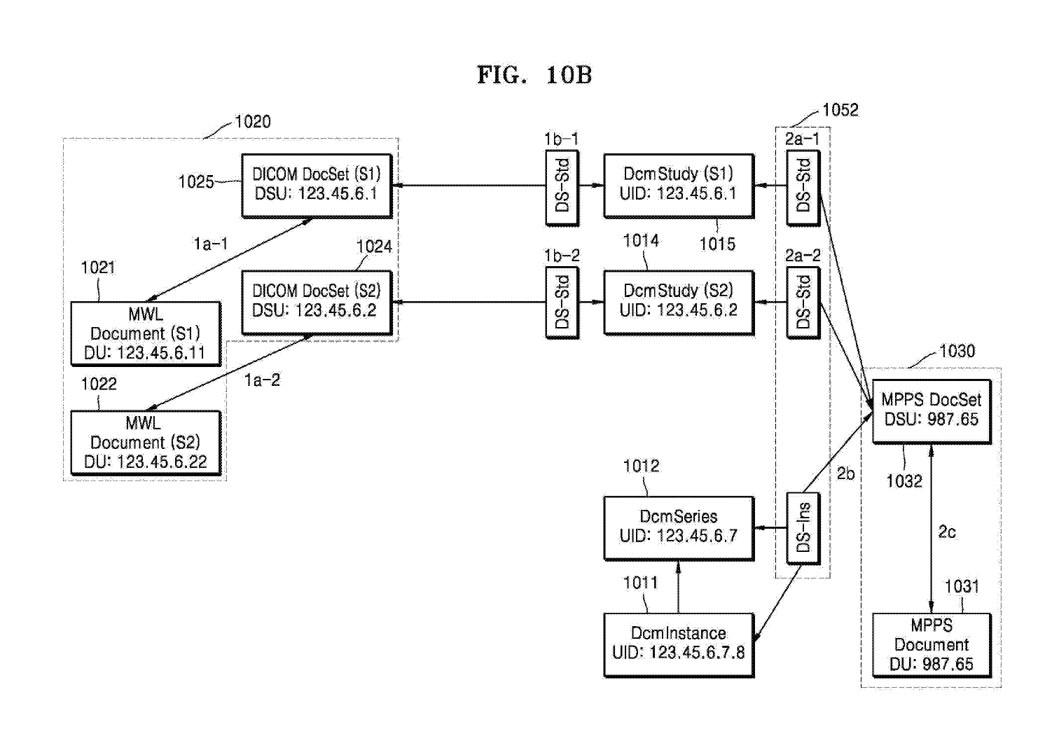

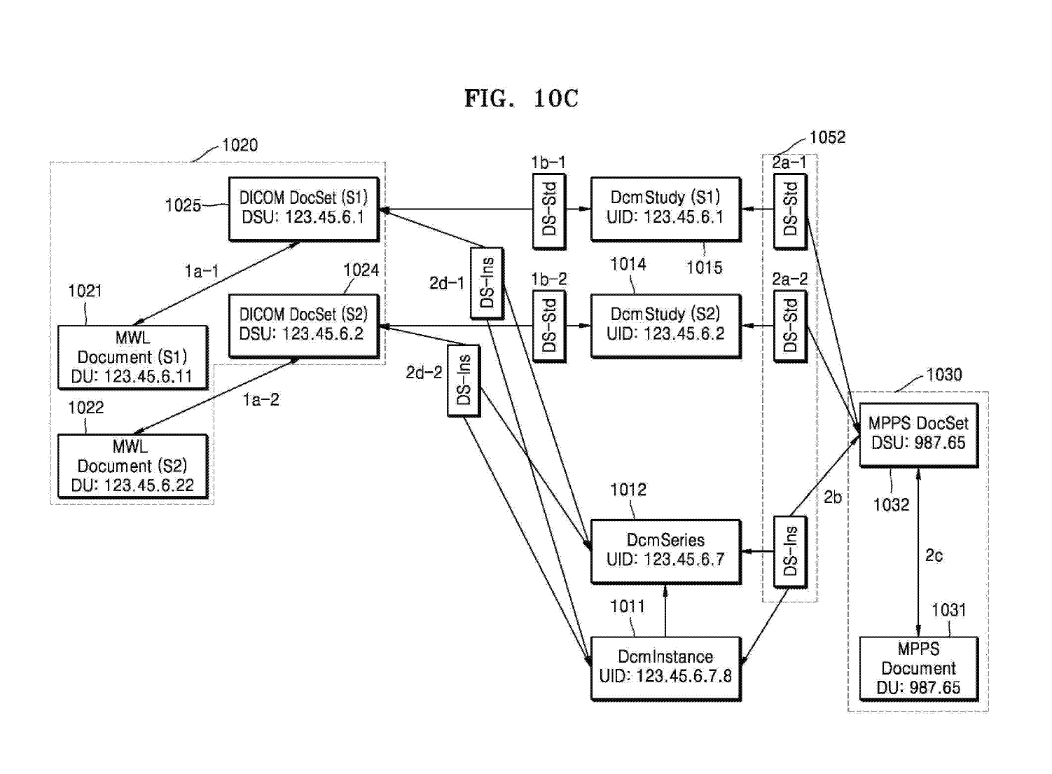

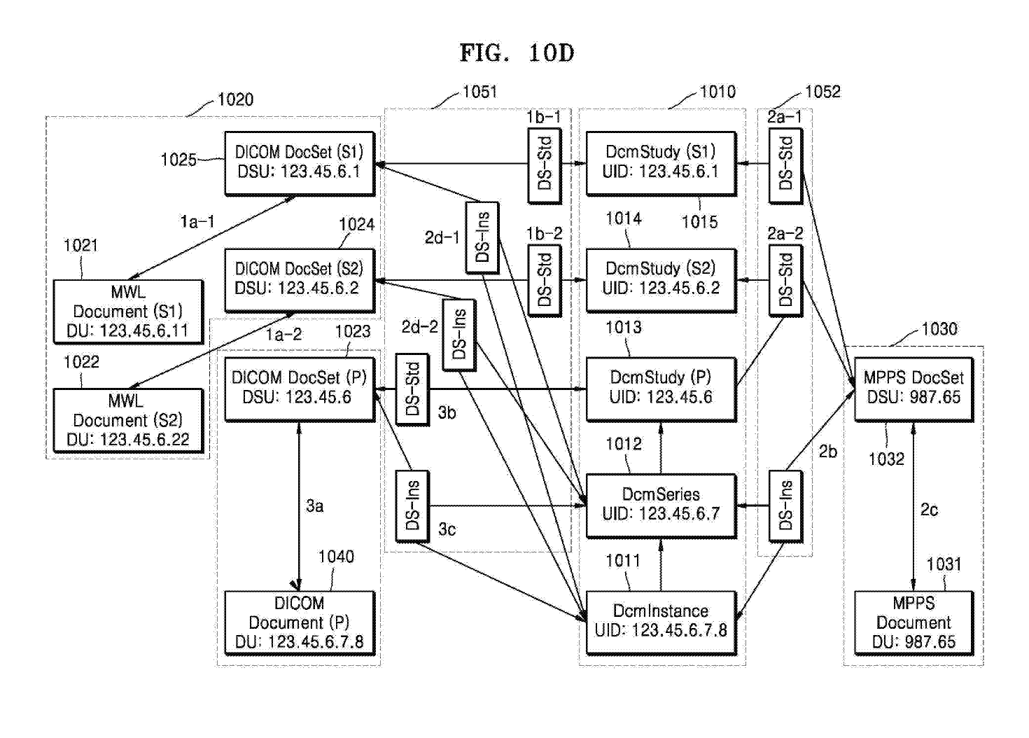

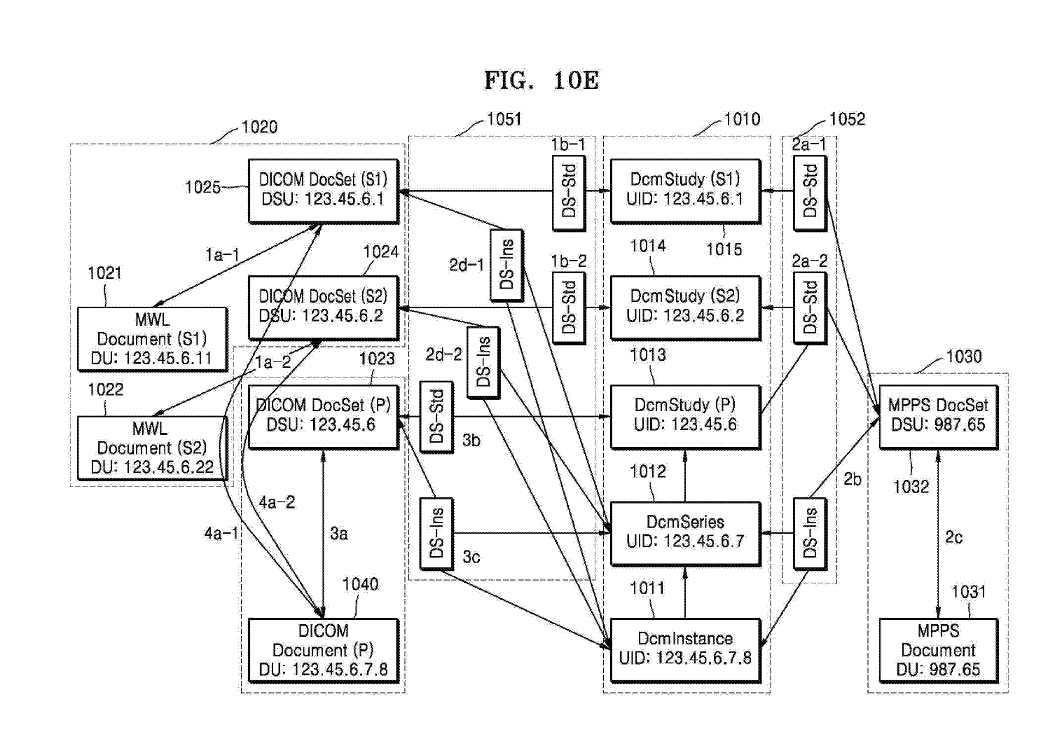

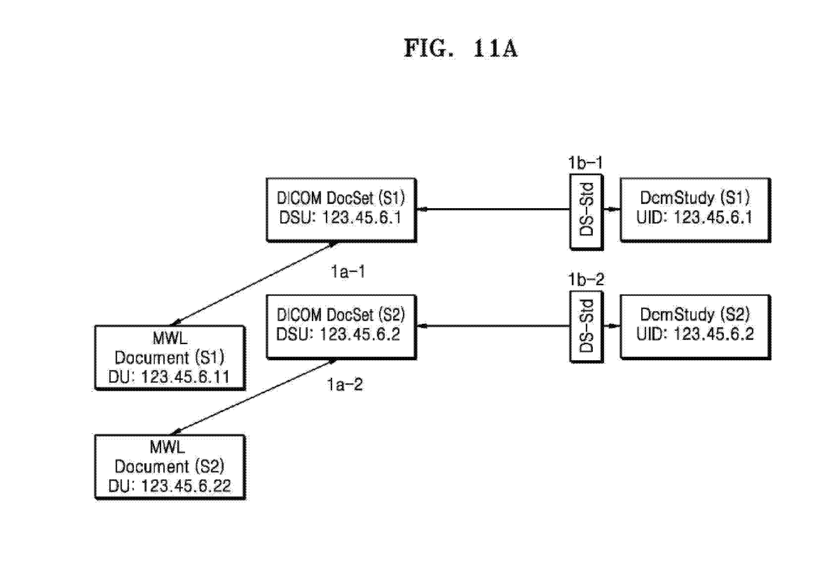

Hereinafter, a basic concept in which the apparatus for generating medical data 10 generates the link data so that the medical data is linked will be described, by referring to FIG. 3. Also, examples in which the apparatus for generating medical data 10 generates the link data based on the first manipulation type will be described, by referring to FIGS. 4A through 7B. Also, examples in which the apparatus for generating medical data 10 generates the link data based on the second manipulation type will be described, by referring to FIGS. 8A through 9B. Also, examples in which the apparatus for generating medical data 10 generates the link data based on the fourth manipulation type will be described, by referring to FIGS. 10A through 14D.

FIG. 3 is a diagram for describing an example in which the apparatus for generating medical data 10 generates the link data.

FIG. 3 illustrates a relationship among the medical data generated by the apparatus for generating medical data 10.

Referring to FIG. 3, medical data 310 may include medical data 311 corresponding to an image, medical data 312 corresponding to a series, and medical data 313 corresponding to an examination. The apparatus for generating medical data 10 may assign UID to each of the medical data 311, the medical data 312, and the medical data 313.

For example, the medical data 311 may be a DcmInstance table or object generated by using DICOM Instance information. Here, a unique key of the medical data 311 may include SOP Instance UID or a complex key based on the SOP Instance UID. Also, the medical data 312 may be a DcmSeries table or object generated by using DICOM Series information. Here, a unique key of the medical data 312 may include Series Instance UID or a complex key based on the Series Instance UID. Also, the medical data 313 may be a DcmStudy table or object generated by using DICOM Study information. Here, a unique key of the medical data 313 may include Study Instance UID or a complex key based on the Study Instance UID.

The apparatus for generating medical data 10 may generate the link data for linking the medical data generated by the electronic medical record system 20, the medical imaging device 30, and the picture archiving communication system 40. Also, as described above with reference to FIG. 1, the apparatus for generating medical data 10 may generate the link data for linking not only the medical data generated by the electronic medical record system 20, the medical imaging device 30, and the picture archiving communication system 40 included in the medical system 1, but also the medical data generated by the radiology information system and the laboratory information system.

For example, medical data 320 of FIG. 3 may include medical data 321 generated by the electronic medical record system 20 and medical data 323 generated by the medical imaging device 30.

For example, the electronic medical record system 20 may command the medical imaging device 30 to execute an examination. In other words, the electronic medical record system 20 may generate the medical data 321 to transmit at least one order to the medical imaging device 30. Hereinafter, the transmission of the order from the electronic medical record system 20 to the medical imaging device 30 will be described as a “modality worklist (MWL).” The meaning of the MWL is as described in the IHE medical information standard.

The apparatus for generating medical data 10 may assign a unique document class DC and document UID DU to each of the medical data 321 and 323. Also, the apparatus for generating medical data 10 may assign a unique DocSet class DSC and DocSet UID DSU to medical data 322 corresponding to a DocSet including the medical data 321 and 323.

For example, the medical data 321 may be a Document table or object generated by using DICOM MWL information or HL7 OPM/OMI information. Here, a unique key of the medical data 321 may be a unique value generated based on Study Instance UID. Also, the medical data 322 may be a DocSet table or object generated by using DICOM Study information. Here, a unique key of the medical data 322 may include the Study Instance UID or a complex key based on the Study Instance UID. Also, the medical data 323 may be a Document table or object generated by using DICOM Instance information. Here, a unique key of the medical data 323 may include SOP Instance UID or a complex key based on the SOP Instance UID.

As another example, medical data 331 included in medical data 330 of FIG. 3 may be medical data including information to be transmitted by the medical imaging device 30 to the electronic medical record system 20 and/or the picture archiving communication system 40. For example, the medical imaging device 30 may report information related to execution of an examination to the electronic medical record system 20 and/or the picture archiving communication system 40. In other words, the medical imaging device 30 may report to the electronic medical record system 20 and/or the picture archiving communication system 40 that the medical imaging device 30 has captured at least one medical image. Also, the medical imaging device 30 may request the picture archiving communication system 40 to store the at least one captured medical image. Hereinafter, the reporting of the medical imaging device 30 to the electronic medical record system 20 and/or the picture archiving communication system 40 that the medical image has been captured will be referred to as a “modality performed procedure step (MPPS).” Also, the requesting the picture archiving communication system 40 to store at least one captured medical image, the requesting performed by the medical imaging device 30, will be referred to as “Storage.” The meanings of the MPPS and the Storage are as described in the IHE medical information standard.

The apparatus for generating medical data 10 may assign a unique document class DC and document UID DU to the medical data 331. Also, the apparatus for generating medical data 10 may assign a unique DocSet class DSC and DocSet UID DSU to medical data 332 corresponding to a DocSet including the medical data 331.

For example, the medical data 331 may be a Document table or object generated by using DICOM MPPS information. Here, a unique key of the medical data 331 may include MPPS Instance UID or a complex key based on the MPPS Instance UID. Also, the medical data 332 may be a DocSet table or object generated by using the DICOM MPPS information. Here, a unique key of the medical data 332 may include the MPPS Instance UID or the complex key based on the MPPS Instance UID.

Also, the apparatus for generating medical data 10 may generate link data 341 and 342 to link the medical data 310, 320, and 330. In other words, the apparatus for generating medical data 10 may generate the link data 341 and 342 to link the medical data 310, 320, and 330 based on a predetermined rule. For example, the link data 341 and 342 may be a table or an object storing link information.

The medical data 310, the medical data 320, and the medical data 330 are data each generated by a different device or system, and thus, they may not be executed by other devices or systems. For example, the medical data 321 may not be executed by the medical imaging device 30. Thus, information (for example, an order) to be transmitted by the electronic medical record system 20 may not be accurately transmitted to the medical imaging device 30, via the medical data 321. The apparatus for generating medical data 10 may generate link data 341 and 342 to link the medical data 310, 320, and 330, based on a manipulation type, so that the medical data 310, 320, and 330 may be compatible with one another. Thus, information (for example, an order) to be transmitted by a first subject may be accurately transmitted to a second subject.

Hereinafter, examples in which the apparatus for generating medical data 10 generates the link data based on the manipulation type will be described by referring to FIGS. 4A through 14D. Meanwhile, document UID, document class DC, DocSet UID DSU, and DocSet class DSC assigned to each of medical data illustrated in FIGS. 4A through 14D are the same as described above with reference to FIGS. 1 through 3, and thus, they will not be described.

FIGS. 4A through 7B are diagrams for describing examples in which the apparatus for generating medical data 10 generates the link data based on the first manipulation type.

FIGS. 4A through 4C illustrate an example in which the apparatus for generating medical data 10 operates in a case in which medical data is transmitted and received in an order of MWL→MPPS→Storage. In detail, FIG. 4A illustrates an example in which DICOM DocSet, MWL Document, and DcmStudy objects 422, 421, and 413 are generated by using DICOM MWL information. Also, FIG. 4B illustrates an example in which MPPS DocSet, DcmSeries, DcmInstance, and MPPS Document objects 432, 412, 411, and 431, are generated by using DICOM MPPS information. Also, FIG. 4C illustrates an example in which a DICOM Document object 440 is generated by using DICOM Composite object information.

Referring to FIG. 4A, the apparatus for generating medical data 10 may generate a link 1a between the DICOM DocSet object 422 and the MWL Document object 421. Also, the apparatus for generating medical data 10 may generate a link 1b between the DICOM DocSet object 422 and the DcmStudy object 413.

Also, referring to FIG. 4B, the apparatus for generating medical data 10 may generate a link 2a between the MPPS DocSet object 432 and the DcmStudy object 413. Also, the apparatus for generating medical data 10 may generate a link 2b among the MPPS DocSet object 432, the DcmSeries object 412, and the DcmInstance object 411. Also, the apparatus for generating medical data 10 may generate a link 2c between the MPPS DocSet object 432 and the MPPS Document object 431.

Also, referring to FIG. 4C, the apparatus for generating medical data 10 may generate a link 3a between the DICOM DocSet object 422 and the DICOM Document object 440. Also, the apparatus for generating medical data 10 may generate a link 3b among the DICOM DocSet object 422, the DcmSeries object 412, and the DcmInstance object 411.

In other words, FIGS. 4A through 4C illustrate medical data in a case in which when the electronic medical record system 20 transmits a single order to the medical imaging device 30, the medical imaging device 30 reports to the electronic medical record system 20 and/or the picture archiving communication system 40 that at least one medical image has been captured, and the medical imaging device 30 requests the picture archiving communication system 40 to store the at least one captured medical image.

The electronic medical record system 20 may generate the medical data 421 related to an MWL. In detail, the electronic medical record system 20 may generate the medical data 421 corresponding to an MWL document, and the apparatus for generating medical data 10 may assign document UID DU to the medical data 421. Also, the apparatus for generating medical data 10 may generate the medical data 422 corresponding to a DocSet including the medical data 421 and assign DocSet UID DSU to the medical data 422.

Also, the apparatus for generating medical data 10 may generate medical data 410 corresponding to at least one medical image captured by the medical imaging device 30. In detail, the apparatus for generating medical data 10 may generate the medical data 411 corresponding to a medical image, the medical data 412 corresponding to a series, and the medical data 413 corresponding to an examination, and may assign UID to each of the medical data 411, 412, and 413.

Also, the medical imaging device 30 may generate the medical data 431 related to an MPPS. In detail, the medical imaging device 30 may generate the medical data 431 corresponding to an MPPS document, and the apparatus for generating medical data 10 may assign document UID DU to the medical data 431. Also, the apparatus for generating medical data 10 may generate the medical data 432 corresponding to a DocSet including the medical data 4341 and assign DocSet UID DSU to the medical data 432.

Also, the apparatus for generating medical data 10 may assign document UID to the medical data 440 related to Storage.

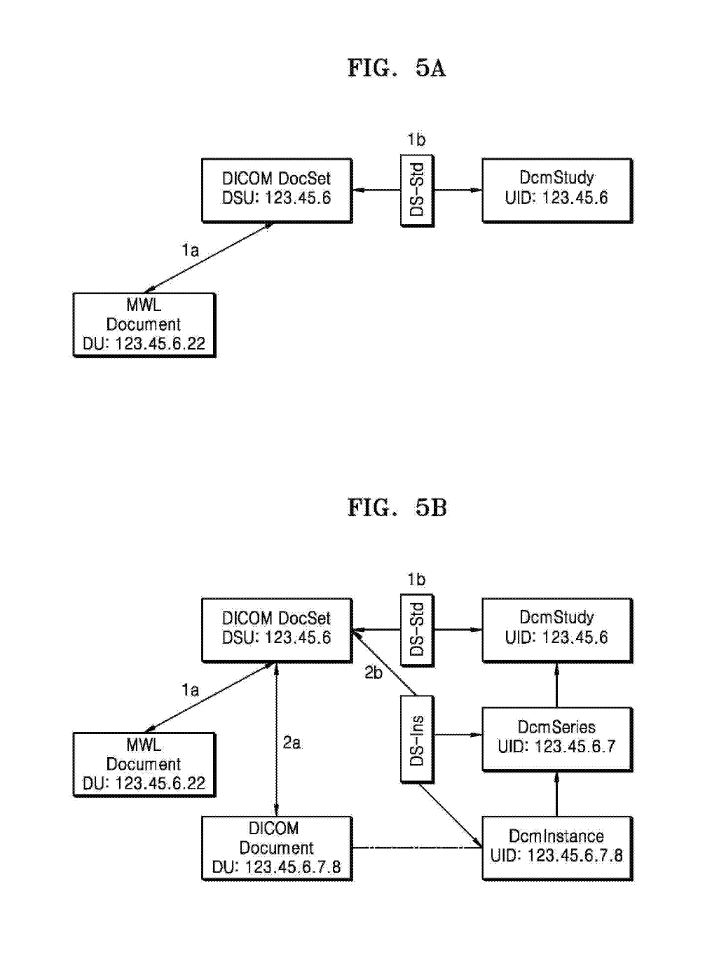

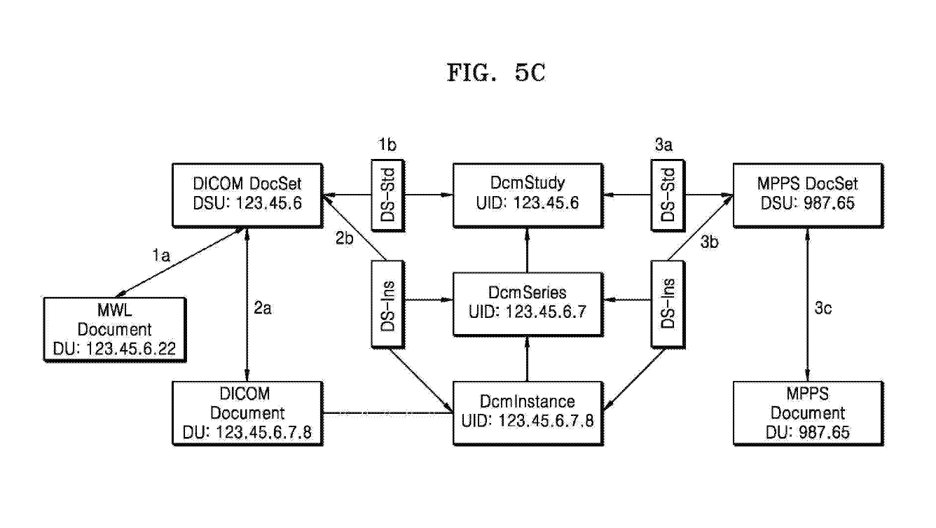

Also, the apparatus for generating medical data 10 may generate link data 451 and 452 to link the medical data 410, 420, 430, and 440 based on a predetermined rule. FIGS. 5A through 5C illustrate an example in which the apparatus for generating medical data 10 operates in a case in which medical data is transmitted and received in an order of MWL→Storage→MPPS. In detail, FIG. 5A illustrates an example in which DICOM DocSet, MWL Document, and DcmStudy objects are generated by using DICOM MWL information. Also, FIG. 5B illustrates an example in which DICOM Document, DcmSeries, and DcmInstance objects are generated by using DICOM Composite object information. Also, FIG. 5C illustrates an example in which MPPS DocSet and MPPS Document objects are generated by using DICOM MPPS information.

Referring to FIG. 5A, the apparatus for generating medical data 10 may generate a link 1a between the DICOM DocSet object and the MWL Document object. Also, the apparatus for generating medical data 10 may generate a link 1b between the DICOM DocSet object and the DcmStudy object.

Also, referring to FIG. 5B, the apparatus for generating medical data 10 may generate a link 2a between the DICOM DocSet object and the DICOM Document object. Also, the apparatus for generating medical data 10 may generate a link 2b among the DICOM DocSet object, the DcmSeries object, and the DcmInstance object.

Also, referring to FIG. 5C, the apparatus for generating medical data 10 may generate a link 3a between the MPPS DocSet object and the DcmStudy object. Also, the apparatus for generating medical data 10 may generate a link 3b among the MPPS DocSet object, the DcmSeries object, and the DcmInstance object. Also, the apparatus for generating medical data 10 may generate a link 3c between the MPPS DocSet object and the MPPS Document object.

In other words, FIGS. 5A through 5C illustrate medical data in a case in which when the electronic medical record system 20 transmits a single order to the medical imaging device 30, the medical imaging device 30 requests the picture archiving communication system 40 to store a captured medical image, and the medical imaging device 30 reports to the electronic medical record system 20 and/or the picture archiving communication system 40 that at least one medical image has been captured.

Thus, the medical data illustrated in FIGS. 5A through 5C is the same as the medical data illustrated in FIGS. 4A through 4C, and is different from the medical data illustrated in FIGS. 4A through 4C only in terms of a time point in which the medical data is generated. Thus, hereinafter, the medical data illustrated in FIGS. 5A through 5C will not be described in detail.

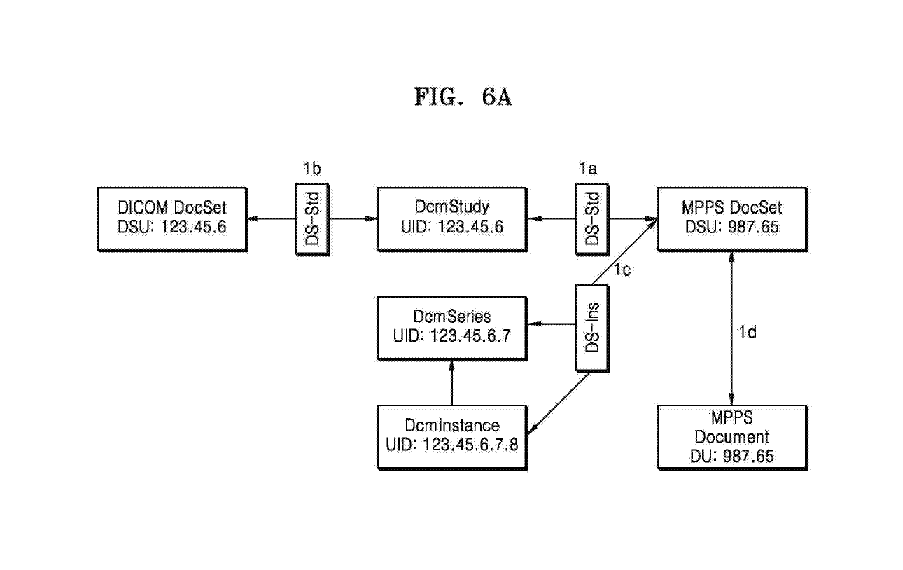

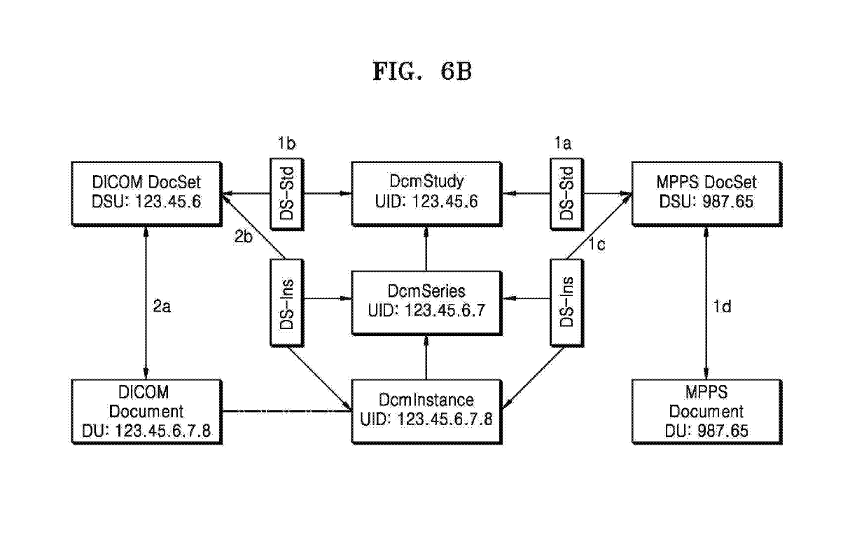

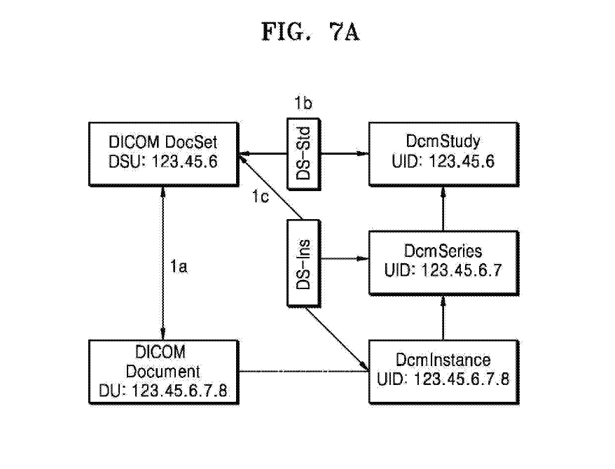

FIGS. 6A and 6B illustrate an example in which the apparatus for generating medical data 10 operates in a case in which medical data is transmitted and received in an order of MPPS→Storage. In detail, FIG. 6A illustrates an example in which MPPS DocSet, DcmStudy, DcmSeries, DcmInstance, MPPS Document, and DICOM DocSet objects are generated by using DICOM MPPS information. Also, FIG. 6B illustrates an example in which a DICOM Document object is generated by using DICOM Composite object information.

Referring to FIG. 6A, the apparatus for generating medical data 10 may generate a link 1a between the MPPS DocSet object and the DcmStudy object. Also, the apparatus for generating medical data 10 may generate a link 1b between the DICOM DocSet object and the DcmStudy object. Also, the apparatus for generating medical data 10 may generate a link 1c among the MPPS DocSet object, the DcmSeries object, and the DcmInstance object. Also, the apparatus for generating medical data 10 may generate a link 1d between the MPPS DocSet object and the MPPS Document object.

Also, referring to FIG. 6B, the apparatus for generating medical data 10 may generate a link 2a between the DICOM DocSet object and the DICOM Document object. Also, the apparatus for generating medical data 10 may generate a link 2b among the DICOM DocSet object, the DcmSeries object, and the DcmInstance object.

In other words, FIGS. 6A and 6B illustrate medical data in a case in which the medical imaging device 30 reports to the electronic medical record system 20 and/or the picture archiving communication system 40 that at least one medical image has been captured, and the medical imaging device 30 requests the picture archiving communication system 40 to store the at least one captured medical image.

Thus, the medical data illustrated in FIGS. 6A and 6B is the same as the medical data 410, 420, 430, and 440 illustrated in FIGS. 4A through 4C, excluding only the medical data 421 related to the MWL. In other words, FIGS. 4A through 4C illustrate the example in which the medical data is transmitted and received in the order of MWL→MPPS→Storage, and FIGS. 6A and 6B illustrate the example in which the medical data is transmitted and received in the order of MPPS→Storage. Thus, although FIGS. 6A and 6B do not explicitly illustrate the medical data 421 related to the MWL, the medical data 421 related to the MWL may be indirectly generated via the MPPS or the Storage. Thus, hereinafter, the medical data illustrated in FIGS. 6A and 6B will not be described in detail.

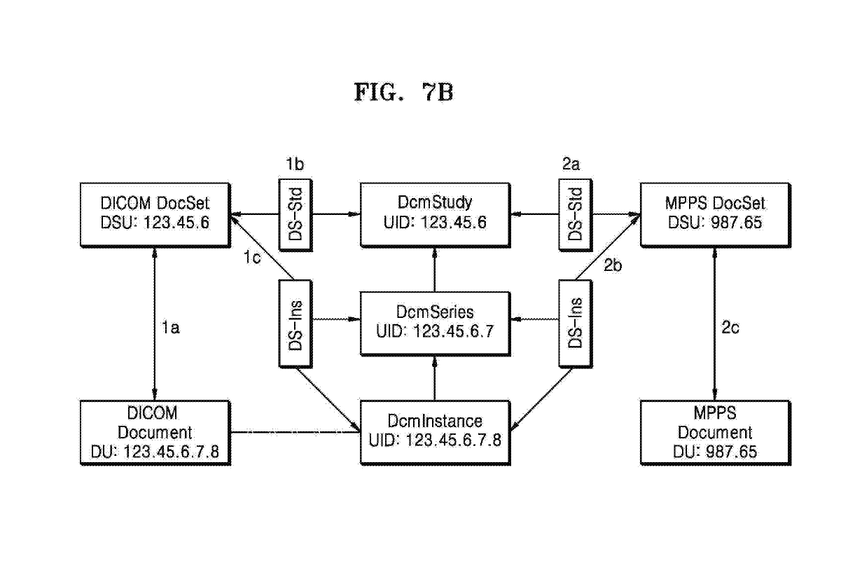

FIGS. 7A and 7B illustrate an example in which the apparatus for generating medical data 10 operates in a case in which medical data is transmitted and received in an order of Storage→MPPS. In detail, FIG. 7A illustrates an example in which DICOM DocSet, DICOM Document, DcmStudy, DcmSeries, and DcmInstance objects are generated by using DICOM Composite object information. Also, FIG. 7B illustrates an example in which MPPS DocSet and MPPS Document objects are generated by using DICOM MPPS information.

Referring to FIG. 7A, the apparatus for generating medical data 10 may generate a link 1a between the DICOM DocSet object and the DICOM Document object. Also, the apparatus for generating medical data 10 may generate a link 1b between the DICOM DocSet object and the DcmStudy object. Also, the apparatus for generating medical data 10 may generate a link 1c among the DICOM DocSet object, the DcmSeries object, and the DcmInstance object.

Also, referring to FIG. 7B, the apparatus for generating medical data 10 may generate a link 2a between the MPPS DocSet object and the DcmStudy object. Also, the apparatus for generating medical data 10 may generate a link 2b among the MPPS DocSet object, the DcmSeries object, and the DcmInstance object. Also, the apparatus for generating medical data 10 may generate a link 2c between the MPPS DocSet object and the MPPS Document object.

In other words, FIGS. 7A and 7B illustrate medical data in a case in which the medical imaging device 30 requests the picture archiving communication system 40 to store a captured medical image and reports to the electronic medical record system 20 and/or the picture archiving communication system 40 that at least one medical image has been captured.

Thus, the medical data illustrated in FIGS. 7A and 7B is the same as the medical data illustrated in FIGS. 6A and 6B, and is different from the medical data illustrated in FIGS. 6A and 6B only in terms of a time point in which the medical data is generated. In other words, FIGS. 6A and 6B illustrate the example in which the medical data is transmitted and received in the order of MPPS→Storage, and FIGS. 7A and 7B illustrate the example in which the medical data is transmitted and received in the order of Storage→MPPS. Thus, as described above with reference to FIGS. 6A and 6B, although FIGS. 7A and 7B do not explicitly illustrate the medical data 421 related to the MWL, the medical data 421 related to the MWL may be indirectly generated via the MPPS or the Storage. Thus, hereinafter, the medical data illustrated in FIGS. 7A and 7B will not be described in detail.

FIGS. 8A through 9B are diagrams for describing examples in which the apparatus for generating medical data 10 generates the link data based on the second manipulation type, according to an embodiment.

FIGS. 8A and 8B illustrate an example in which the apparatus for generating medical data 10 operates in a case in which medical data is transmitted and received in an order of MPPS→Storage. In detail, FIG. 8A illustrates an example in which MPPS DocSet, DcmSeries, DcmInstance, MPPS Document, and DICOM DocSet objects are generated by using DICOM MPPS information. Also, FIG. 8B illustrates an example in which DICOM DocSet, DICOM Document, and DcmStudy objects are generated by using DICOM Composite object information.

Referring to FIG. 8A, the apparatus for generating medical data 10 may generate a link 1a among the MPPS DocSet object, the DcmSeries object, and the DcmInstance object. Also, the apparatus for generating medical data 10 may generate a link 1b between the MPPS DocSet object and the MPPS Document object.

Also, referring to FIG. 8B, the apparatus for generating medical data 10 may generate a link 2a between the DICOM DocSet object and the DICOM Document object. Also, the apparatus for generating medical data 10 may generate a link 2b between the DICOM DocSet object and the DcmStudy object. Also, the apparatus for generating medical data 10 may generate a link 2c among the DICOM DocSet object, the DcmSeries object, and the DcmInstance object.

In other words, FIGS. 8A and 8B illustrate medical data in a case in which the medical imaging device 30 reports to the electronic medical record system 20 and/or the picture archiving communication system 40 that at least one medical image has been captured, and the medical imaging device 30 requests the picture archiving communication system 40 to store the at least one captured medical image.

Thus, the medical data illustrated in FIGS. 8A and 8B is the same as the medical data 410, 420, 430, and 440 illustrated in FIGS. 4A through 4C, excluding the medical data 421 related to the MWL. The medical data illustrated in FIGS. 8A and 8B is different from the medical data illustrated in FIGS. 6A and 6B and the medical data illustrated in FIGS. 7A and 7B in that while the medical data illustrated in FIGS. 6A and 6B and the medical data illustrated in FIGS. 7A and 7B are examples in which the medical data 421 related to the MWL may be indirectly generated via the MPPS or the Storage, the medical data illustrated in FIGS. 8A and 8B is an example in which the medical data 421 related to the MWL may not be generated. Thus, hereinafter, the medical data illustrated in FIGS. 8A and 8B will not be described in detail.

FIGS. 9A and 9B illustrate an example in which the apparatus for generating medical data 10 operates in a case in which medical data is transmitted and received in an order of Storage→MPPS. In detail, FIG. 9A illustrates an example in which DICOM DocSet, DICOM Document, DcmStudy, DcmSeries, and DcmInstance objects are generated by using DICOM Composite object information. Also, FIG. 9B illustrates an example in which MPPS DocSet and MPPS Document objects are generated by using DICOM MPPS information.

Referring to FIG. 9A, the apparatus for generating medical data 10 may generate a link 1a between the DICOM DocSet object and the DICOM document object. Also, the apparatus for generating medical data 10 may generate a link 1b between the DICOM DocSet object and the DcmStudy object. Also, the apparatus for generating medical data 10 may generate a link 1c among the DICOM DocSet object, the DcmSeries object, and the DcmInstance object.

Also, referring to FIG. 9B, the apparatus for generating medical data 10 may generate a link 2a among the MPPS DocSet object, the DcmSeries object, and the DcmInstance object. Also, the apparatus for generating medical data 10 may generate a link 2b between the MPPS DocSet object and the MPPS Document object.

In other words, FIGS. 9A and 9B illustrate medical data in a case in which the medical imaging device 30 requests the picture archiving communication system 40 to store a captured medical image and reports to the electronic medical record system 20 and/or the picture archiving communication system 40 that at least one medical image has been captured.

Thus, the medical data illustrated in FIGS. 9A and 9B is the same as the medical data illustrated in FIGS. 8A and 8B, and is different from the medical data illustrated in FIGS. 8A and 8B only in terms of a time point in which the medical data is generated. In other words, FIGS. 8A and 8B illustrate the example in which the medical data is transmitted and received in the order of MPPS→Storage, and FIGS. 9A and 9B illustrate the example in which the medical data is transmitted and received in the order of Storage→MPPS. Thus, as described above in detail with reference to FIGS. 8A and 8B, the examples illustrated in FIGS. 9A and 9B are examples in which the medical data 421 related to the MWL is not generated. Thus, the medical data illustrated in FIGS. 9A and 9B will not be described in detail.

FIGS. 10A through 14D are diagrams for describing examples in which the apparatus for generating medical data 10 generates the link data based on the fourth manipulation type, according to an embodiment.

FIGS. 10A through 10E illustrate an example in which the apparatus for generating medical data 10 operates in a case in which medical data is transmitted and received in an order of MWL→MPPS→Storage. In detail, FIG. 10A illustrates an example in which DICOM DocSet (Sn), MWL Document (Sn), and DcmStudy (Sn) object sets 1024 and 1025, 1021 and 1022, and 1014 and 1015 are generated by using a plurality of pieces of DICOM MWL information. Here, S denotes Scheduled, and n denotes a number for identifying a plurality of MWLs. Also, FIGS. 10B and 100 illustrate an example in which MPPS DocSet, DcmSeries, DcmInstance, and MPPS Document objects 1032, 1012, 1011, and 1031 are generated by using DICOM MPPS information. Also, FIG. 10D illustrates an example in which DICOM DocSet (P), DICOM Document (P), and DcmStudy (P) objects, 1023, 1040, and 1013, different from the DICOM DocSet (Sn), MWL Document (Sn), and DcmStudy (Sn) objects generated in FIG. 10A, are generated by using DICOM Composite object information. Here, P denotes performed. Also, FIG. 10E illustrates an example in which a link is generated between the DICOM DocSet (Sn) object and the DICOM Document (P) object by using the DICOM Composite object information and the DICOM MPPS information.

Referring to FIG. 10A, the apparatus for generating medical data 10 may generate links 1a-1 and 1a-2 between the DICOM DocSet (Sn) objects 1024 and 1025 and the MWL Document (Sn) objects 1021 and 1022. Also, the apparatus for generating medical data 10 may generate links 1b-1 and 1b-2 between the DICOM DocSet (Sn) objects 1024 and 1025 and the DcmStudy (Sn) objects 1014 and 1015.

Also, referring to FIG. 10B, the apparatus for generating medical data 10 may generate links 2a-1 and 2a-2 between the MPPS DocSet object 1032 and the plurality of DcmStudy (Sn) objects 1014 and 1015, respectively. Also, the apparatus for generating medical data 10 may generate a link 2b among the MPPS DocSet object 1032, the DcmSeries object 1012, and the DcmInstance object 1011. Also, the apparatus for generating medical data 10 may generate a link 2c between the MPPS DocSet object 1032 and the MPPS Document object 1031.

Also, referring to FIG. 100, the apparatus for generating medical data 10 may generate links 2d-1 and 2d-2 among the DICOM DocSet (Sn) objects 1024 and 1025, the DcmSeries object 1012, and the DcmInstance object 1011.

Also, referring to FIG. 10D, the apparatus for generating medical data 10 may generate a link 3a between the DICOM DocSet (P) object 1023 and the DICOM Document (P) object 1040. Also, the apparatus for generating medical data 10 may generate a link 3b between the DICOM DocSet (P) object 1023 and the DcmStudy (P) object 1013. Also, the apparatus for generating medical data 10 may generate a link 3c among the DICOM DocSet (P) object 1023, the DcmSeries object 1012, and the DcmInstance object 1011.

Also, referring to FIG. 10E, the apparatus for generating medical data 10 may generate links 4a-1 and 4a-2 between the DICOM DocSet (Sn) objects 1024 and 1025 and the DICOM Document (P) object 1040, respectively, by using the DICOM composite object information and the DICOM MPPS information.

In other words, FIGS. 10A through 10E illustrate medical data in a case in which when the electronic medical record system 20 transmits a plurality of orders to the medical imaging device 30, the medical imaging device 30 reports to the electronic medical record system 20 and/or the picture archiving communication system 40 that a single examination has been executed (that is, at least one medical image has been captured), and the medical imaging device 30 requests the picture archiving communication system 40 to store the at least one captured medical image.

The electronic medical record system 20 may generate the medical data 1021 and 1022 related to an MWL. In detail, the electronic medical record system 20 may generate the medical data 1021 and 1022 corresponding to a plurality of MWL documents, and the apparatus for generating medical data 10 may assign document UID DU to the medical data 1021 and 1022. Also, the apparatus for generating medical data 10 may generate the medical data 1025 corresponding to a DocSet including the medical data 1021, and generate the medical data 1024 corresponding to a DocSet including the medical data 1022. Also, the apparatus for generating medical data 10 may assign DocSet UID DSU to the medical data 1024 and 1025.

Also, the apparatus for generating medical data 10 may generate medical data 1010 corresponding to at least one medical image captured by the medical imaging device 30. In detail, the apparatus for generating medical data 10 may generate the medical data 1011 corresponding to a medical image, the medical data 1012 corresponding to a series, and the medical data 1013, 1014, and 1015 corresponding to an examination, and assign UID to each of the medical data 1011, 1012, 1013, 1014, and 1015.

Also, the medical imaging device 30 may generate the medical data 1031 related to an MPPS. In detail, the medical imaging device 30 may generate the medical data 1031 corresponding to an MPPS document and the apparatus for generating medical data 10 may assign document UID DU to the medical data 1031. Also, the apparatus for generating medical data 10 may generate the medical data 1032 corresponding to a DocSet including the medical data 1031, and assign DocSet UID DSU to the medical data 1032.

Also, the apparatus for generating medical data 10 may assign document UID to the medical data 1040 related to Storage. Also, the apparatus for generating medical data 10 may generate the medical data 1023 corresponding to a DocSet including the medical data 1040 and assign DocSet UID DSU to the medical data 1023.

Also, the apparatus for generating medical data 10 may generate link data 1051 and 1052 to link the medical data 1010, 1020, 1030, and 1040 based on a predetermined rule.

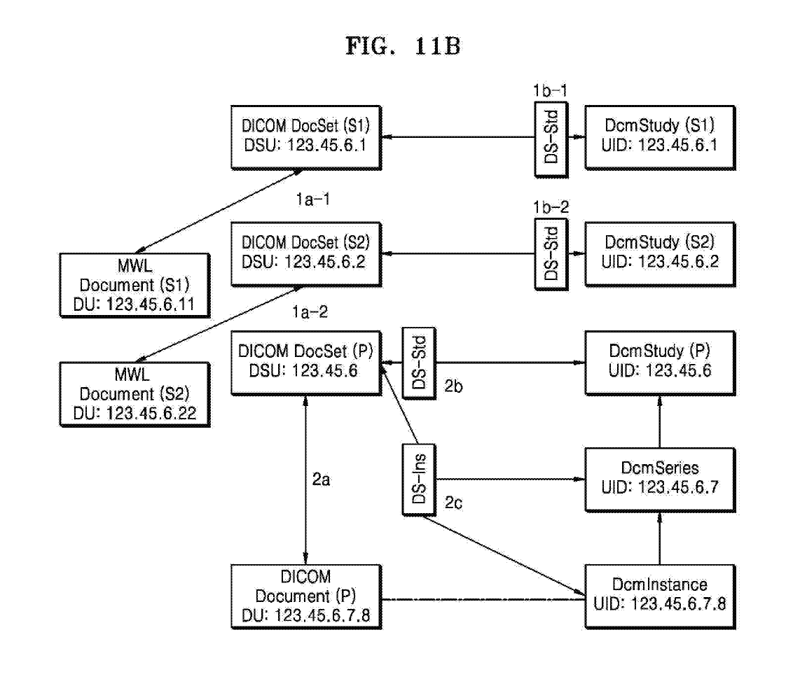

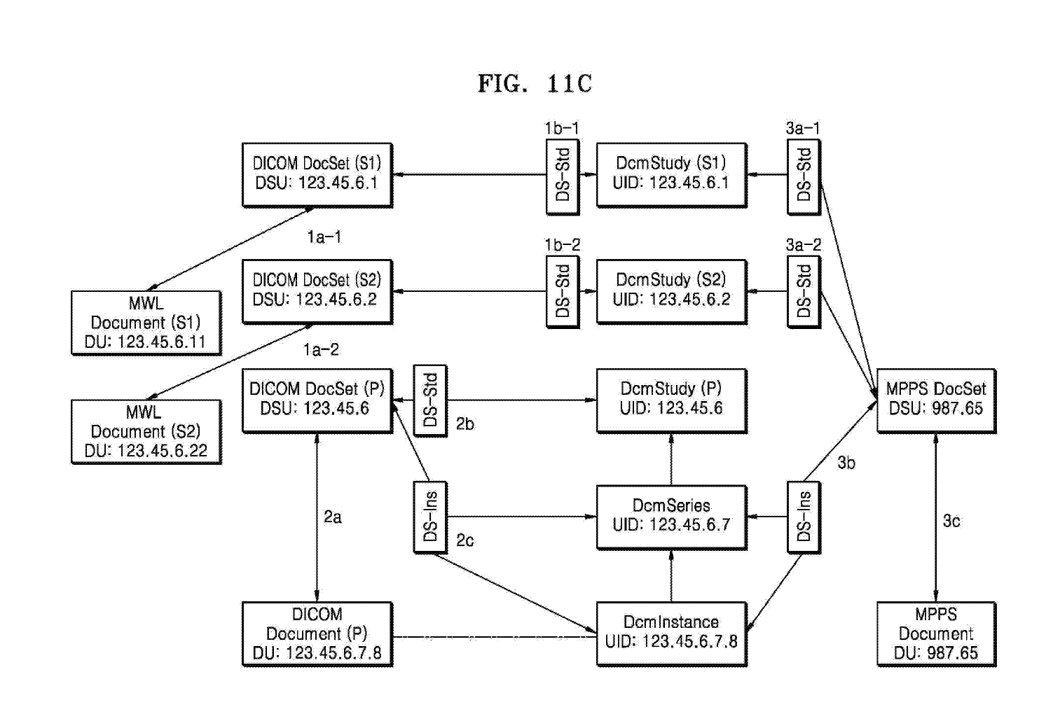

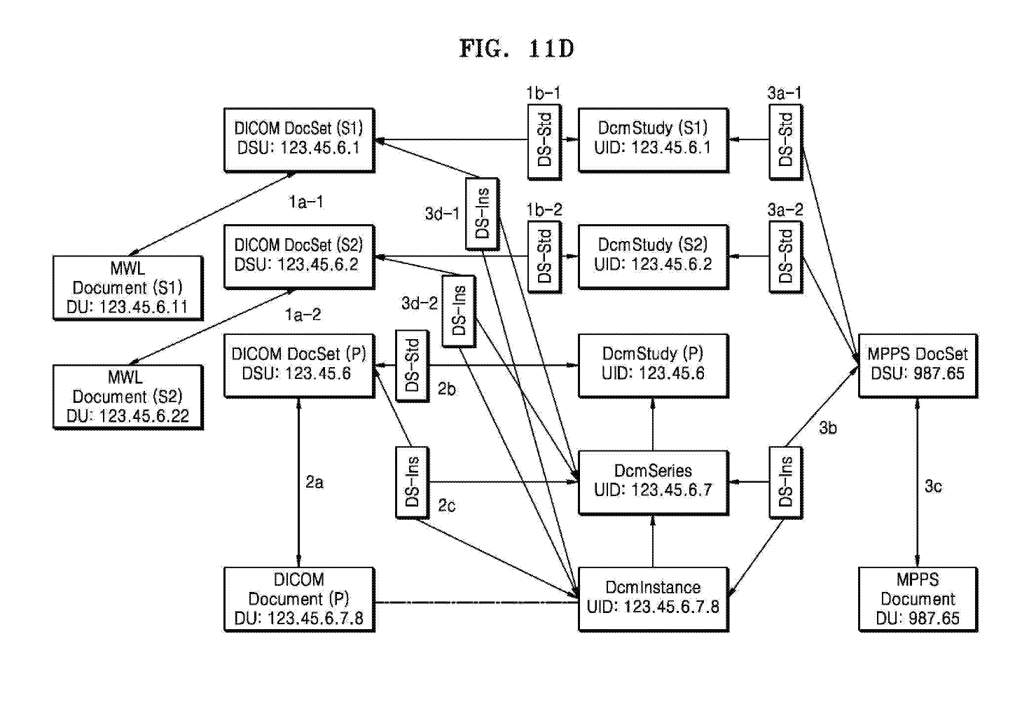

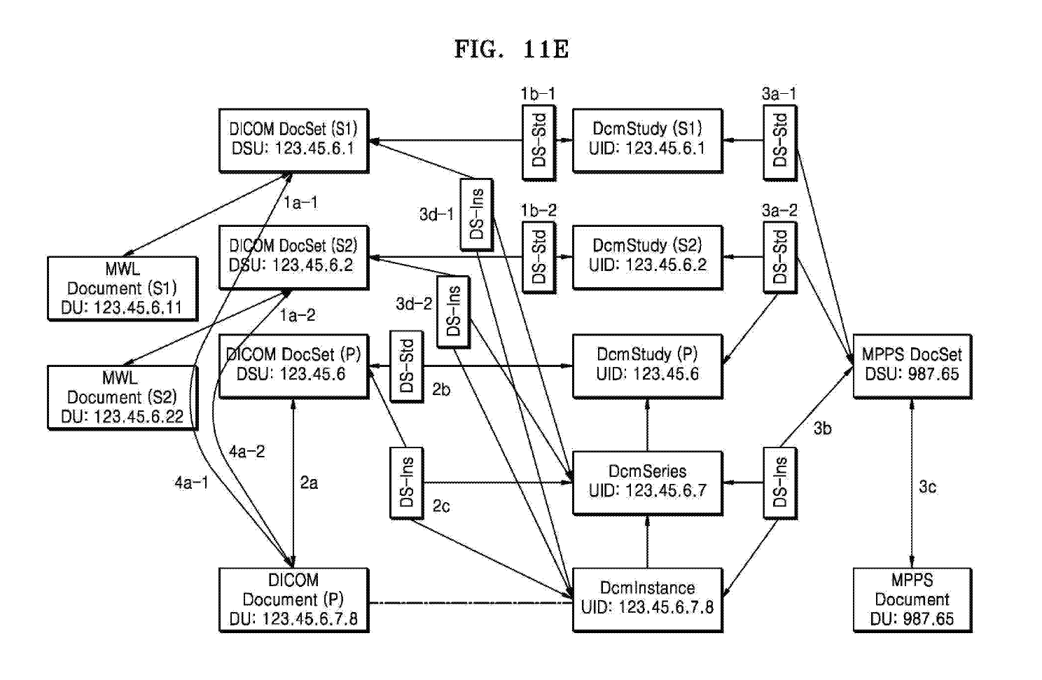

FIGS. 11A through 11E illustrate an example in which the apparatus for generating medical data 10 operates in a case in which medical data is transmitted and received in an order of MWL→Storage→MPPS. In detail, FIG. 11A illustrates an example in which DICOM DocSet (Sn), MWL Document (Sn), and DcmStudy (Sn) object sets are generated by using a plurality of pieces of DICOM MWL information. Here, S denotes Scheduled, and n denotes a number for identifying a plurality of MWLs. Also, FIG. 11B illustrates an example in which DICOM DocSet (P), DICOM Document (P), DcmStudy (P), DcmSeries, and DcmInstance objects, different from the DICOM DocSet (Sn), MWL Document (Sn), and DcmStudy (Sn) object sets generated in FIG. 11A, are generated by using DICOM Composite object information. Here, P denotes Performed. Also, FIGS. 11C and 11D illustrate an example in which MPPS DocSet and MPPS Document objects are generated by using DICOM MPPS information. Also, FIG. 11E illustrates an example in which a link is generated between the DICOM DocSet (Sn) object and the DICOM Document (P) object by using the DICOM Composite object information and the DICOM MPPS information.

Referring to FIG. 11A, the apparatus for generating medical data 10 may generate links 1a-1 and 1a-2 between the DICOM DocSet (Sn) object and the MWL Document (Sn) object. Also, the apparatus for generating medical data 10 may generate links 1b-1 and 1b-2 between the DICOM DocSet (Sn) object and the DcmStudy (Sn) object.

Also, referring to FIG. 11B, the apparatus for generating medical data 10 may generate a link 2a between the DICOM DocSet (P) object and the DICOM Document (P) object. Also, the apparatus for generating medical data 10 may generate a link 2b between the DICOM DocSet (P) object and the DcmStudy (P) object. Also, the apparatus for generating medical data 10 may generate a link 2c among the DICOM DocSet (P) object, the DcmSeries object, and the DcmInstance object.

Also, referring to FIG. 11C, the apparatus for generating medical data 10 may generate links 3a-1 and 3a-2 between the MPPS DocSet object and a plurality of DcmStudy (Sn) objects. Also, the apparatus for generating medical data 10 may generate a link 3b among the MPPS DocSet object, the DcmSeries object, and the DcmInstance object. Also, the apparatus for generating medical data 10 may generate a link 3c between the MPPS DocSet object and the MPPS Document object.

Also, referring to FIG. 11D, the apparatus for generating medical data 10 may generate links 3d-1 and 3d-2 among the DICOM DocSet (Sn) object, the DcmSeries object, and the DcmInstance object.

Also, referring to FIG. 11E, the apparatus for generating medical data 10 may generate links 4a-1 and 4a-2 between the DICOM DocSet (Sn) object and the DICOM Document (P) object by using DICOM composite object information and DICOM MPPS information.

In other words, FIGS. 11A through 11E illustrate medical data in a case in which when the electronic medical record system 20 transmits a plurality of orders to the medical imaging device 30, the medical imaging device 30 requests the picture archiving communication system 40 to store a captured medical image, and the medical imaging device 30 reports to the electronic medical record system 20 and/or the picture archiving communication system 40 that a single examination has been executed (that is, at least one medical image has been captured).

Thus, the medical data illustrated in FIGS. 11A through 11E is the same as the medical data illustrated in FIGS. 10A through 10E, and is different from the medical data illustrated in FIGS. 10A through 10E only in terms of a time point in which the medical data is generated. Thus, hereinafter, the medical data illustrated in FIGS. 11A through 11E will not be described in detail.

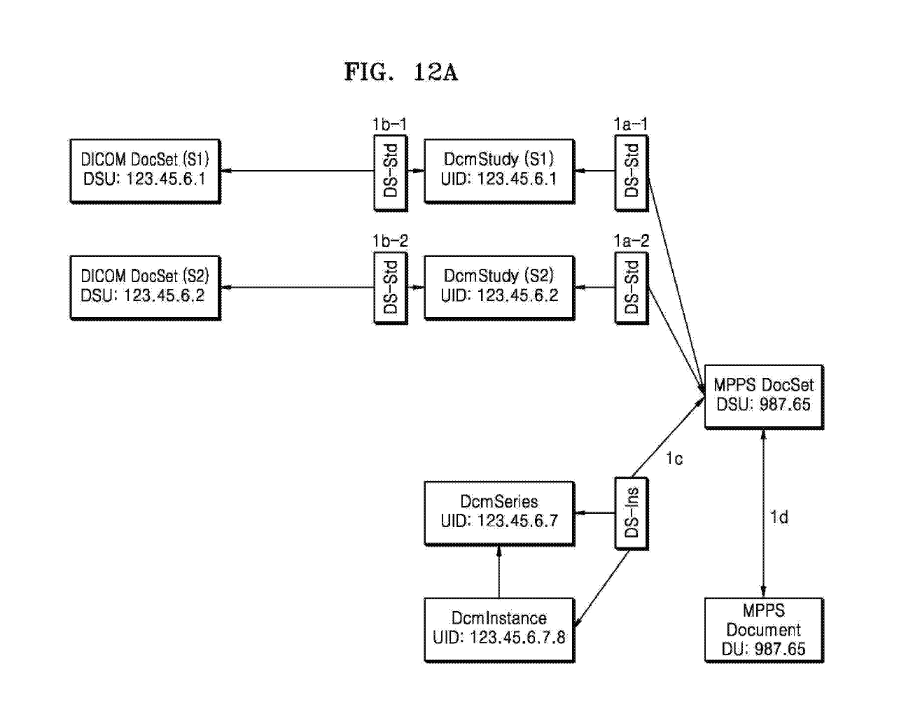

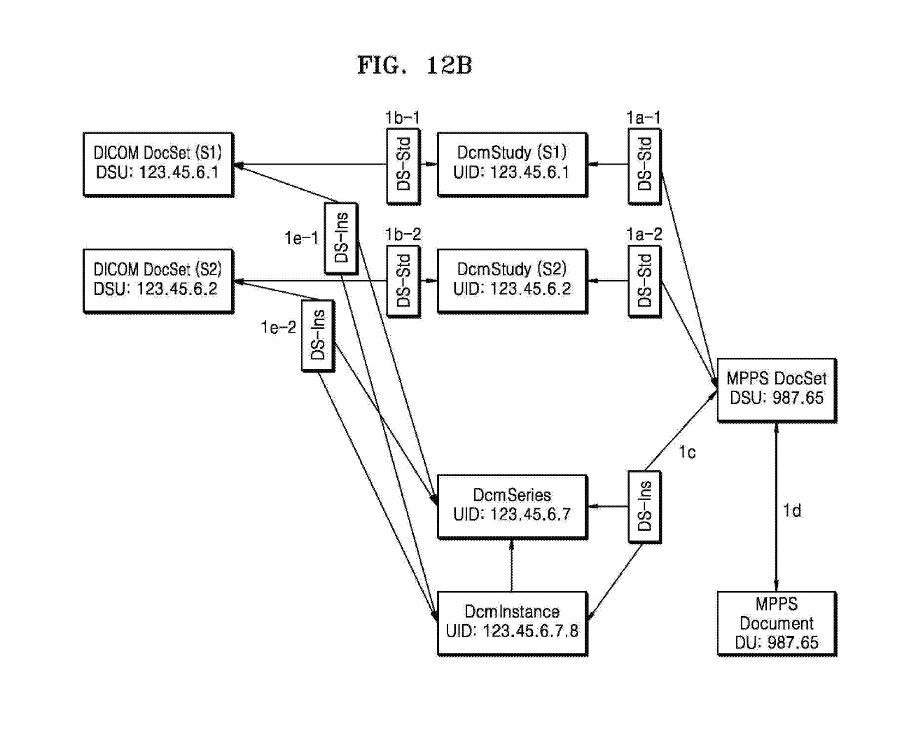

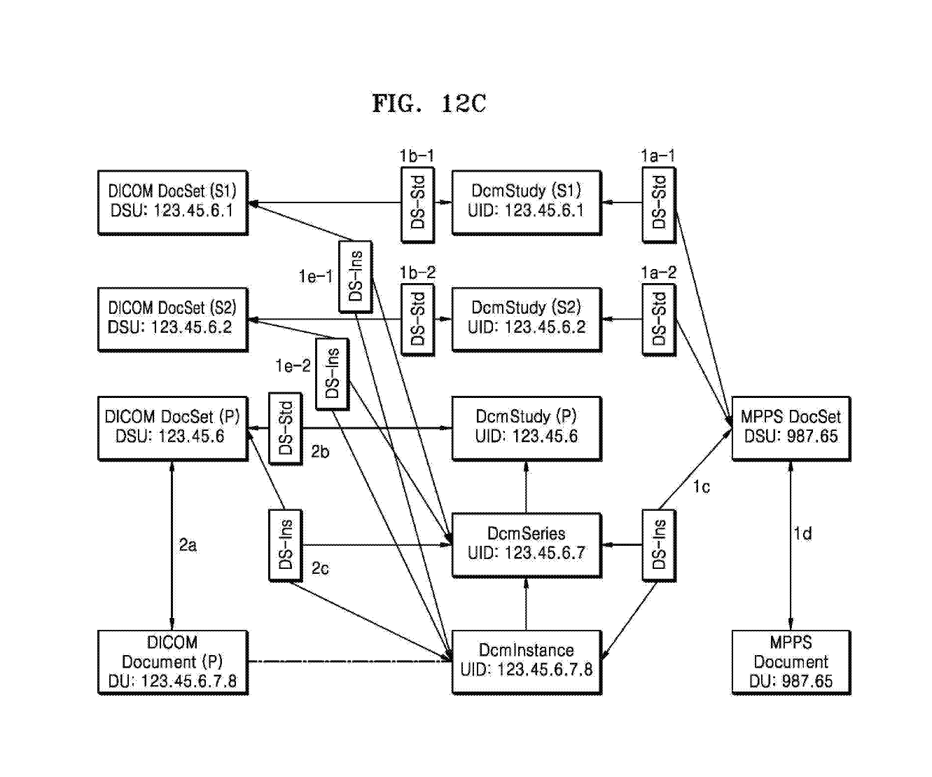

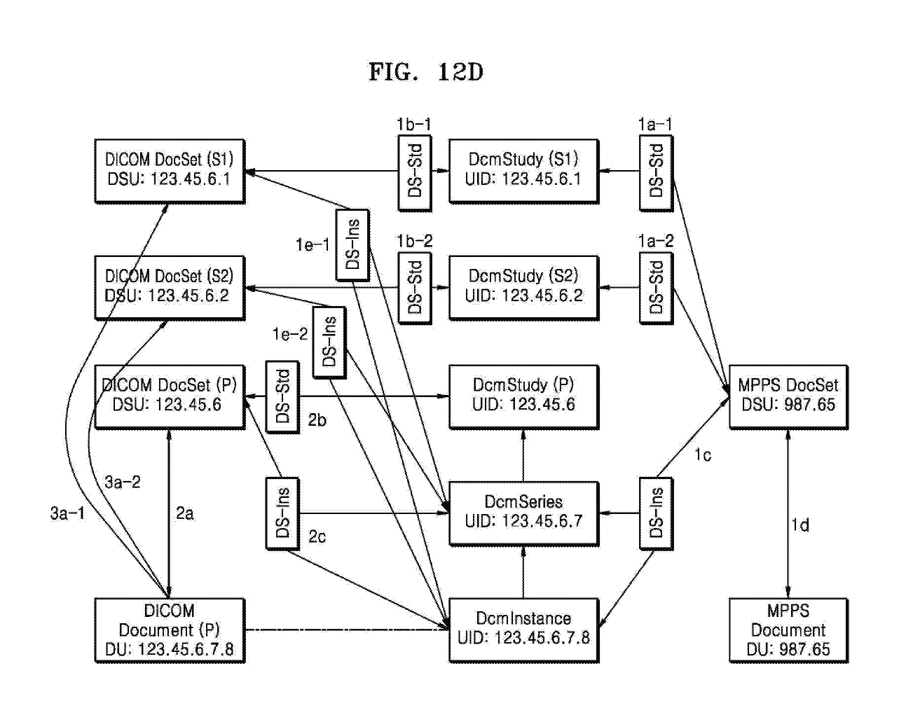

FIGS. 12A through 12D illustrate an example in which the apparatus for generating medical data 10 operates in a case in which medical data is transmitted and received in an order of MPPS→Storage. In detail, FIGS. 12A and 12B illustrate an example in which MPPS DocSet, DcmStudy (Sn), DICOM DocSet (Sn), DcmSeries, DcmInstance, MPPS Document objects are generated by using DICOM MPPS information. Here, S denotes Scheduled, and n denotes a number for identifying a plurality of MWLs. Also, FIG. 12C illustrates an example in which DICOM DocSet (P), DICOM Document (P), and DcmStudy (P) objects, different from the MPPS DocSet, DcmStudy (Sn), DICOM DocSet (Sn), DcmSeries, DcmInstance, and MPPS Document objects illustrated in FIGS. 12A and 12B, are generated by using DICOM Composite object information. Here, P denotes Performed. Also, FIG. 12D illustrates an example in which a link is generated between the DICOM DocSet (Sn) object and the DICOM Document (P) object by using the DICOM composite object information and the DICOM MPPS information.

Referring to FIG. 12A, the apparatus for generating medical data 10 may generate links 1a-1 and 1a-2 between the DICOM DocSet (Sn) object and an MWL Document (Sn) object. Also, the apparatus for generating medical data 10 may generate links 1b-1 and 1b-2 between the DICOM DocSet (Sn) object and the DcmStudy (Sn) object. Also, the apparatus for generating medical data 10 may generate a link 1c among the MPPS DocSet object, the DcmSeries object, and the DcmInstance object. Also, the apparatus for generating medical data 10 may generate a link 1d between the MPPS DocSet object and the MPPS Document object.

Also, referring to FIG. 12B, the apparatus for generating medical data 10 may generate links 1e-1 and 1e-2 among the DICOM DocSet (Sn) object, the DcmSeries object, and the DcmInstance object.

Also, referring to FIG. 12C, the apparatus for generating medical data 10 may generate a link 2a between the DICOM DocSet (P) object and the DICOM Document (P) object. Also, the apparatus for generating medical data 10 may generate a link 2b between the DICOM DocSet (P) object and the DcmStudy (P) object. Also, the apparatus for generating medical data 10 may generate a link 2c among the DICOM DocSet (P) object, the DcmSeries object, and the DcmInstance object.

Also, referring to FIG. 12D, the apparatus for generating medical data 10 may generate links 3a-1 and 3a-2 between the DICOM DocSet (Sn) object and the DICOM Document (P) object by using the DICOM Composite object and the DICOM MPPS information.

In other words, FIGS. 12A through 12D illustrate medical data in a case in which the medical imaging device 30 reports to the electronic medical record system 20 and/or the picture archiving communication system 40 that at least one medical image has been captured, and the medical imaging device 30 requests the picture archiving communication system 40 to store the at least one captured medical image.

Thus, the medical data illustrated in FIGS. 12A through 12D is the same as the medical data 1010, 1020, 1030, and 1040 illustrated in FIGS. 10A through 10E, excluding the medical data 1021 and 1022 related to the MWL. In other words, FIGS. 10A through 10E illustrate the example in which the medical data is transmitted and received in the order of MWL→MPPS→Storage, and FIGS. 12A through 12D illustrate the example in which the medical data is transmitted and received in the order of MPPS→Storage. Thus, although FIGS. 12A through 12D do not explicitly illustrate the medical data 1021 and 1022 related to the MWL, the medical data 1021 and 1022 related to the MWL may be indirectly generated via the MPPS or the Storage. Thus, hereinafter, the medical data illustrated in FIGS. 12A through 12D will not be described in detail.

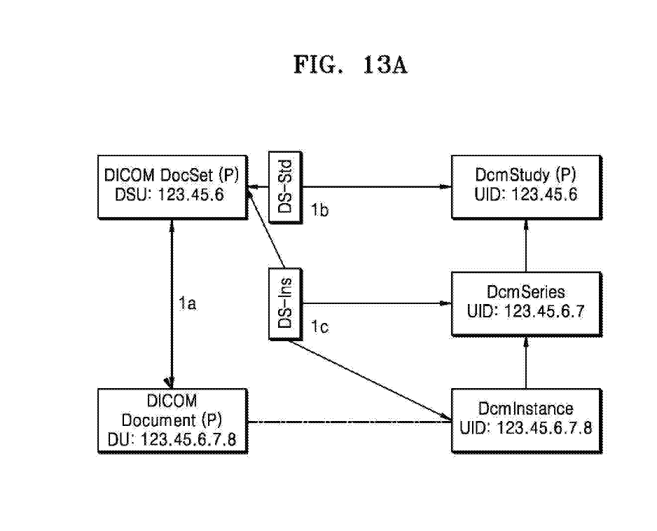

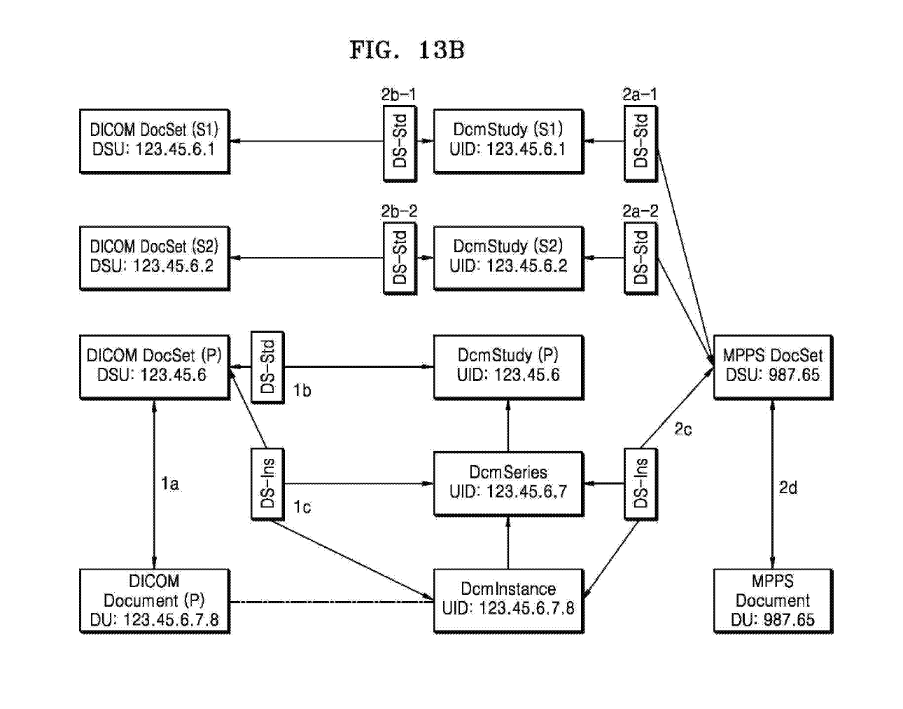

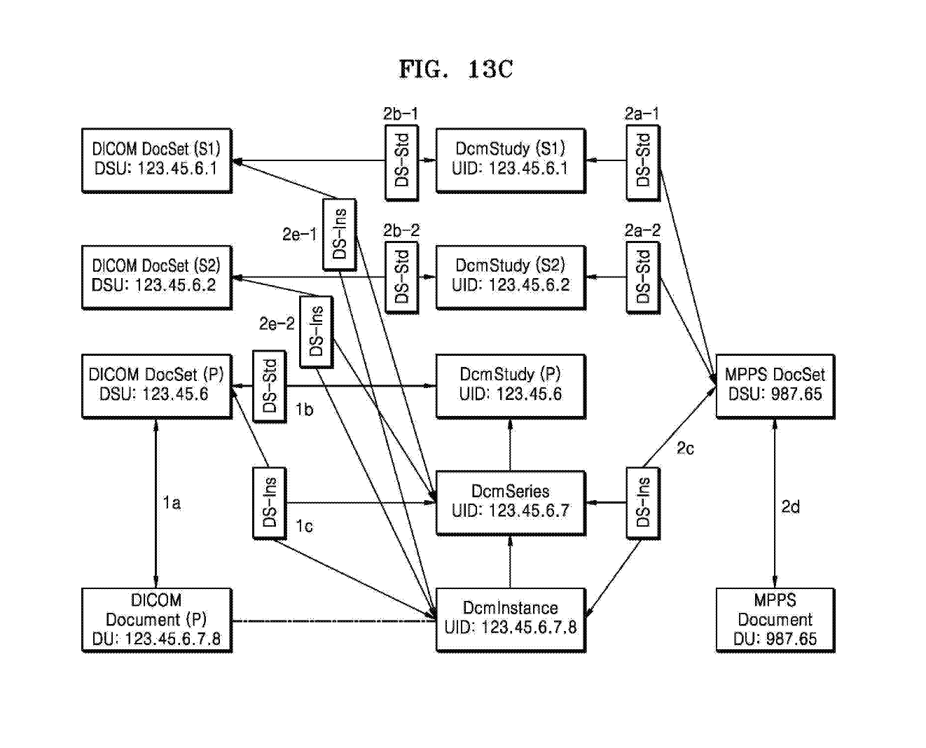

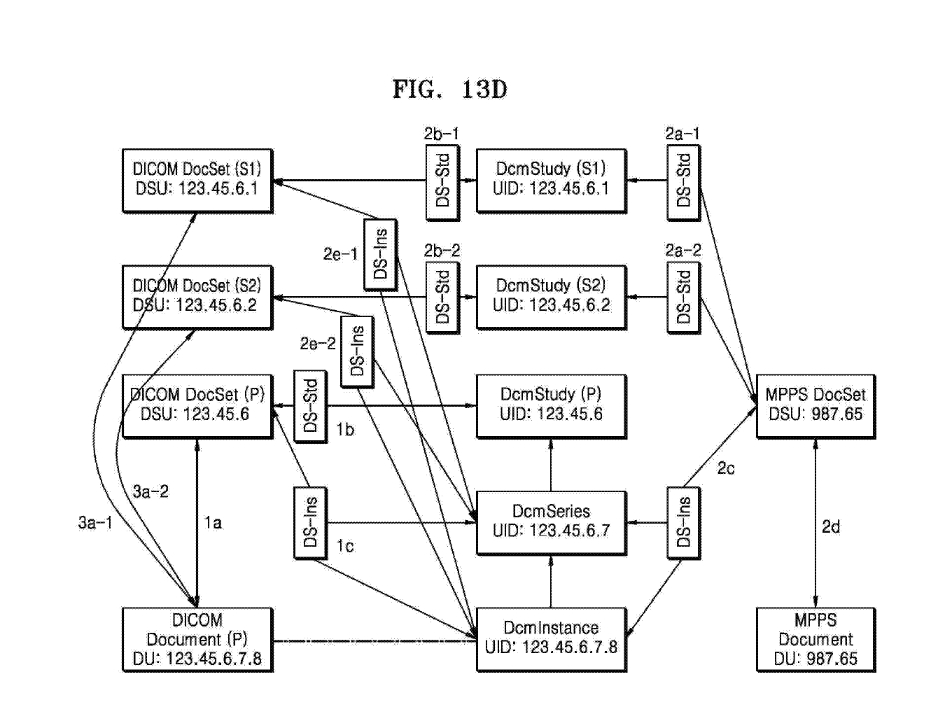

FIGS. 13A through 13D illustrate an example in which the apparatus for generating medical data 10 operates in a case in which medical data is transmitted and received in an order of Storage→MPPS. In detail, FIG. 13A illustrate an example in which DICOM DocSet (P), DICOM Document (P), DcmStudy (P), DcmSeries, and DcmInstance objects are generated by using DICOM Composite object information. Here, S denotes Scheduled. Also, FIGS. 13B and 13C illustrate an example in which MPPS DocSet, MPPS Document, DcmStudy (Sn), and DICOM DocSet (Sn) objects are generated by using DICOM MPPS information. Here, S denotes Scheduled, and n denotes a number for identifying a plurality of MWLs. Also, FIG. 13D illustrates an example in which a link is generated between the DICOM DocSet (Sn) object and the DICOM Document (P) object by using the DICOM Composite object information and the DICOM MPPS information.

Referring to FIG. 13A, the apparatus for generating medical data 10 may generate a link 1a between the DICOM DocSet (P) object and the DICOM Document (P) object. Also, the apparatus for generating medical data 10 may generate a link 1b between the DICOM DocSet (P) object and the DcmStudy (P) object. Also, the apparatus for generating medical data 10 may generate a link 1c among the DICOM DocSet (P) object, the DcmSeries object, and the DcmInstance object.

Also, referring to FIG. 13B, the apparatus for generating medical data 10 may generate links 2a-1 and 2a-2 between the MPPS DocSet object and a plurality of DcmStudy (Sn) objects. Also, the apparatus for generating medical data 10 may generate links 2b-1 and 2b-2 between the DICOM DocSet (Sn) object and the DcmStudy (Sn) object. Also, the apparatus for generating medical data 10 may generate a link 2c among the MPPS DocSet object, the DcmSeries object, and the DcmInstance object. Also, the apparatus for generating medical data 10 may generate a link 2d between the MPPS DocSet object and the MPPS Document object.

Also, referring to FIG. 13C, the apparatus for generating medical data 10 may generate links 2e-1 and 2e-2 among the DICOM DocSet (Sn) object, the DcmSeries object, and the DcmInstance object.

Also, referring to FIG. 13D, the apparatus for generating medical data 10 may generate links 3a-1 and 3a-2 between the DICOM DocSet (Sn) object and the DICOM Document (P) object by using the DICOM Composite object information and the DICOM MPPS information.

In other words, FIGS. 13A through 13D illustrate medical data in a case in which the medical imaging device 30 requests the picture archiving communication system 40 to store a captured medical image, and reports to the electronic medical record system 20 and/or the picture archiving communication system 40 that at least one medical image has been captured.

Thus, the medical data illustrated in FIGS. 13A through 132D is the same as the medical data illustrated in FIGS. 12A through 12D, and is different from the medical data illustrated in FIGS. 12A through 12D only in terms of a time point in which the medical data is generated. In other words, FIGS. 12A through 12D illustrate the example in which the medical data is transmitted and received in the order of MPPS→Storage, and FIGS. 13A through 13D illustrate the example in which the medical data is transmitted and received in the order of Storage→MPPS. Thus, as described in detail above with reference to FIGS. 12A through 12D, although FIGS. 13A through 13D do not explicitly illustrate the medical data 1021 and 1022 related to the MWL, the medical data 1021 and 1022 related to the MWL may be indirectly generated via the MPPS or the Storage. Thus, hereinafter, the medical data illustrated in FIGS. 13A through 13D will not be described in detail.

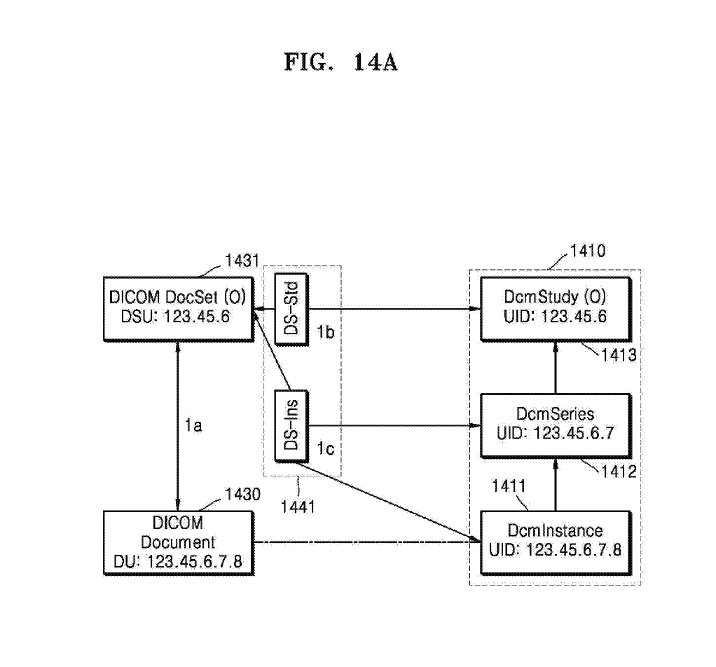

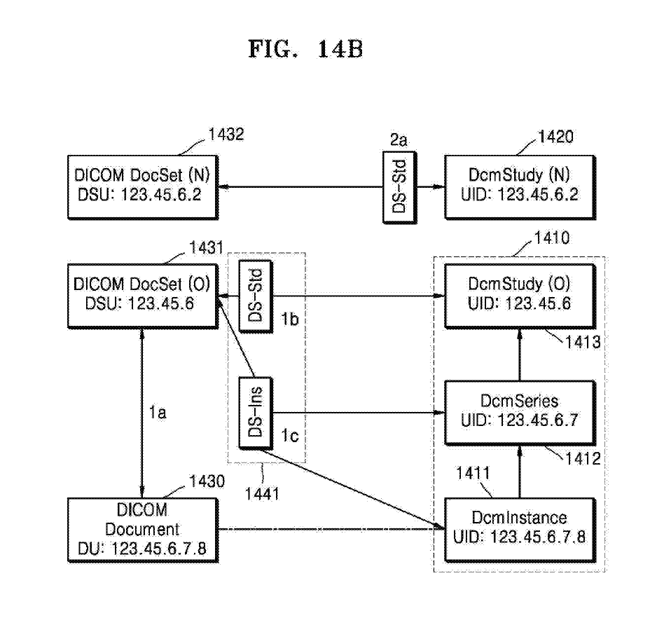

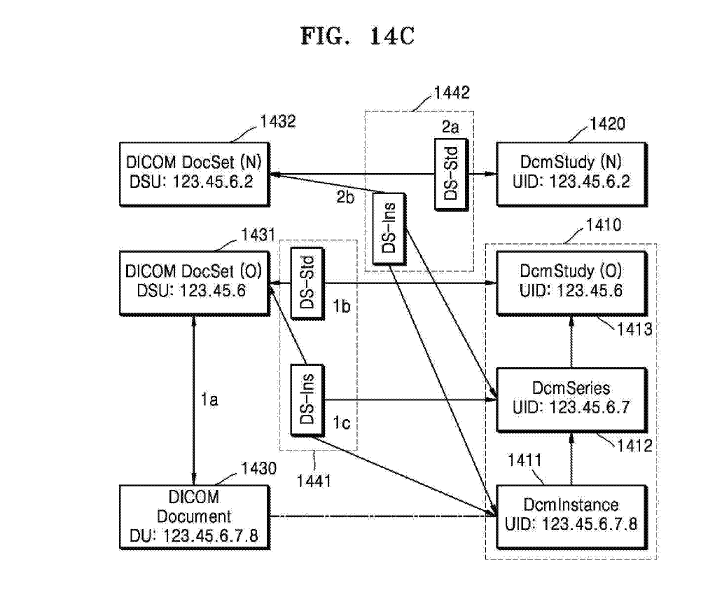

FIGS. 14A through 14D illustrate a case in which medical data is generated when an image is stored and then Study is replicated, divided, and merged. In detail, FIG. 14A illustrate an example in which DICOM DocSet (0), DICOM Document, DcmStudy (0), DcmSeries, and DcmInstance objects 1431, 1430, 1413, 1412, and 1411 are generated by using DICOM Composite object information. Here, 0 denotes Original. Also, FIGS. 14B and 14C illustrate an example in which DICOM DocSet (N) and DcmStudy (N) objects 1432 and 1420 are generated based on a user action, etc. Here, N denotes New. Also, FIG. 14D illustrates an example in which a link is generated between the DICOM DocSet (N) object and the DICOM Document object.

Referring to FIG. 14A, the apparatus for generating medical data 10 may generate a link 1a between the DICOM DocSet (0) object 1431 and the DICOM Document object 1430. Also, the apparatus for generating medical data 10 may generate a link 1b between the DICOM DocSet (0) object 1431 and the DcmStudy (0) object 1413. Also, the apparatus for generating medical data 10 may generate a link 1c among the DICOM DocSet (0) object 1431, the DcmSeries object 1412, and the DcmInstance object 1411.

Also, referring to FIG. 14B, the apparatus for generating medical data 10 may generate a link 2a between the DICOM DocSet (N) object 1432 and the DcmStudy (N) object 1420.

Also, referring to FIG. 14C, the apparatus for generating medical data 10 may generate a link 2b among the DICOM DocSet (N) object 1432, the DcmSeries object 1412, and the DcmInstance object 1411.

Also, referring to FIG. 14D, the apparatus for generating medical data 10 may generate a link 3a between the DICOM DocSet (N) object 1432 and the DICOM Document object 1430.

The DICOM DocSet (N) object 1432 may be linked to the DcmSeries object 1412 and the DcmInstance object 1411 via the link 2b, and to the DICOM Document object 1430 via the link 3a, so that an effect of replicating the Study may be obtained. Here, when a part of the previous link 1c is removed, an effect of dividing the Study may be obtained. As described above, an effect of merging the Study may be obtained based on the manipulation of the links.

In other words, FIGS. 14A through 14D illustrate medical data in a case in which the medical imaging device 30 requests the picture archiving communication system 40 to store images corresponding to an examination, and the medical imaging device 30 replicates, divides, or merges the images corresponding to the examination into a plurality of examinations.

For example, the apparatus for generating medical data 10 may generate medical data 1410 corresponding to at least one medical image captured by the medical imaging device 30. In detail, the apparatus for generating medical data 10 may generate the medical data 1411 corresponding to a medical image, the medical data 1412 corresponding to a series, and the medical data 1413 corresponding to an examination, and assign UID to each of the medical data 1411, 1412, and 1413. Also, the apparatus for generating medical data 10 may assign document UID to the medical data 1430 related to the Storage. Also, the apparatus for generating medical data 10 may generate the medical data 1431 corresponding to a DocSet including the medical data 1030 and assign DocSet UID DSU to the medical data 1431.

The medical data 1431 of FIGS. 14A through 14D is linked to the medical data 1410 corresponding to the examination before division, and the medical data 1432 is linked to the medical data 1420 corresponding to an examination newly generated based on the division. In other words, the apparatus for generating medical data 10 may generate link data 1441 to link the medical data 1410 and 1431, and link data 1442 to link the medical data 1420 and 1432.

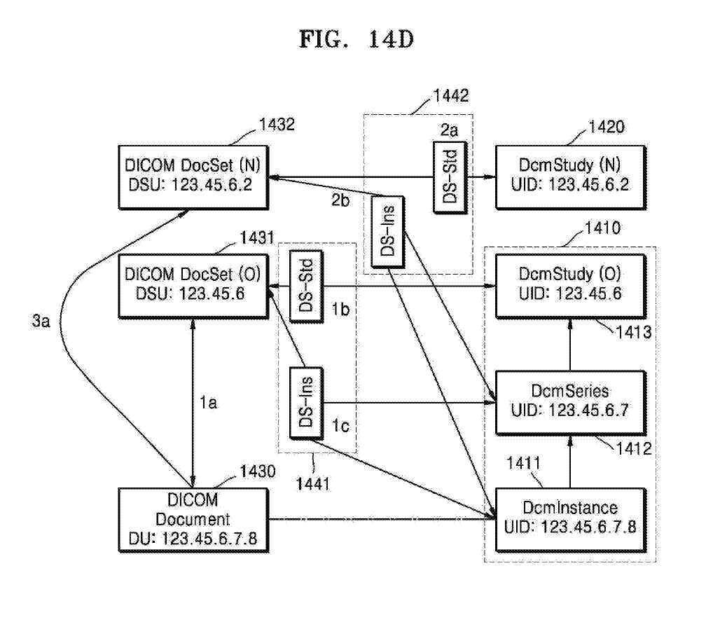

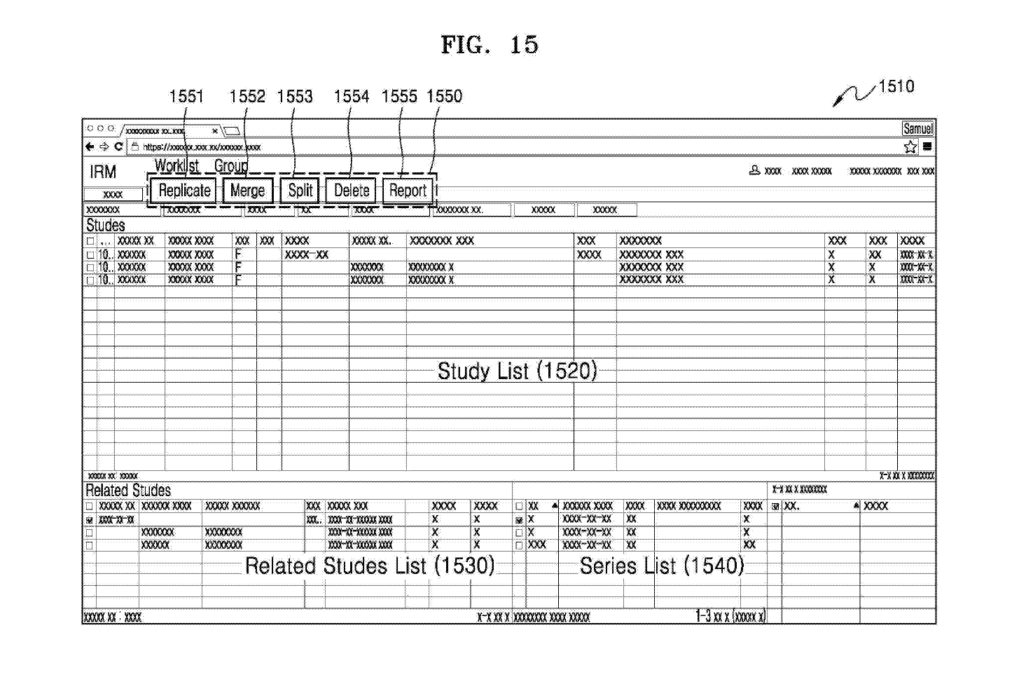

FIG. 15 is a view of an example of a screen 1510 on which medical data is output, according to an embodiment.

Referring to FIG. 15, the screen 1510 may include an area 1520 on which at least one examination list indicating a subject to be examined (that is, a patient) is output, an area 1530 on which an examination list related to an examination indicating the subject to be examined is output, and an area 1540 on which at least one series list corresponding to the examination indicating the subject to be examined is output.

The screen 1510 may display a list 1550 of operations performed by a user based on an examination. For example, the user may replicate a desired examination. For example, when the user selects a replication button 1551 displayed on the screen 1510, the apparatus for generating medical data 10 may replicate the examination designated by the user. As another example, the user may merge two or more examinations into one examination. For example, when the user selects a merge button 1552 displayed on the screen 1510, the examinations designated by the user may be merged. Here, a list of the examinations designated by the user may be output on the screen 1510 via a pop-up window, and the user may select a representative examination from the list displayed on the pop-up window. When the user selects the representative examination, the apparatus for generating medical data 10 may merge the remaining examinations displayed on the pop-up window into the representative examination.

As another example, the user may divide one examination into a plurality of examinations. For example, when the user selects a division button 1553 displayed on the screen 1510, the examination selected by the user may be divided into a plurality of series. Here, a list of the series of the examination selected by the user may be output via a pop-up window on the screen 1510, and the user may select a series to eliminate from the list displayed on the pop-up window. When the user selects the series to eliminate, the apparatus for generating medical data 10 may generate the selected series as a new examination.

As another example, the user may cancel an examination or a series that the user wishes to cancel. For example, when the user selects a cancel button 1554 displayed on the screen 1510, the examination or the series selected by the user may be canceled. Here, a list of the examinations or the series selected by the user may be output on the screen 1510 via a pop-up window, so that the user may check the examinations or the series to cancel.

As another example, the user may write a report with respect to an examination. For example, when the user selects a report button 1555 displayed on the screen 1510, a pop-up window to write a report may be output on the screen 1510.

As described above, according to the one or more of the above embodiments, medical data related to medical imaging may be automatically generated without intervention of a user. In detail, with respect to an order of capturing a medical image, a captured medical image, and a medical operation, link data for linking data generated by each of medical devices may be automatically generated without intervention of the user.

The method described above can be written as computer programs and can be implemented in general-use digital computers that execute the programs using a computer-readable recording medium. Also, the data structure used in the method may be written to the computer-readable recording medium via various means. Examples of the computer-readable recording medium include magnetic Storage media (e.g., read-only memory (ROM), random-access memory (RAM), universal serial bus (USB), floppy disks, hard disks, etc.), optical recording media (e.g., CD-ROMs, or DVDs), etc.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

1. A method of generating medical data, the method comprising:
determining a manipulation type based on an order of generating at least one medical image;
selecting a first subject and a second subject from among a plurality of medical systems, based on the determined manipulation type; and
generating link data for linking first medical data generated by the first subject with second medical data generated by the second subject.

2. The method of claim 1, wherein the determining the manipulation type comprises determining the manipulation type based on whether or not the order exists and based on a number of the order.

3. The method of claim 2, wherein the determining the manipulation type comprises determining any one of a first manipulation type for executing a single examination in response to a single order, a second manipulation type for executing a single examination without the order, a third manipulation type for executing a plurality of examinations in response to a single order, and a fourth manipulation type for executing a single examination in response to a plurality of orders.

4. The method of claim 1, wherein the order of generating at least one medical image comprises an order of generating at least one medical image indicating a region of interest included in a subject to be examined.

5. The method of claim 1, wherein the selecting the first subject and the second subject comprises selecting the first subject and the second subject from among an electronic medical record system, a medical image generating modality, a picture archiving communication system, a radiology information system, and a laboratory information system.

6. A non-transitory computer-readable recording medium having embodied thereon a program for executing a method on an apparatus, the method comprising:
determining a manipulation type based on an order of generating at least one medical image;
selecting a first subject and a second subject from among a plurality of medical systems, based on the determined manipulation type; and
generating link data for linking first medical data generated by the first subject with second medical data generated by the second subject.

7. An apparatus for generating medical data, the apparatus comprising:
a determination portion configured to determine a manipulation type based on an order of generating at least one medical image;
a selection portion configured to select a first subject and a second subject from among a plurality of medical systems, based on the determined manipulation type; and
a generation portion configured to generate link data for linking first medical data generated by the first subject with second medical data generated by the second subject.

8. The apparatus for generating medical data of claim 7, wherein the determination portion is configured to determine the manipulation type based on whether or not the order exists and based on a number of the order.

9. The apparatus for generating medical data of claim 8, wherein the determination portion is configured to determine any one of a first manipulation type for executing a single examination in response to a single order, a second manipulation type for executing a single examination without the order, a third manipulation type for executing a plurality of examinations in response to a single order, and a fourth manipulation type for executing a single examination in response to a plurality of orders.

10. The apparatus for generating medical data of claim 7, wherein the order comprises an order of generating at least one medical image indicating a region of interest included in a subject to be examined.

11. The apparatus for generating medical data of claim 7, wherein the selection portion is configured to select the first subject and the second subject from among an electronic medical record system, a medical image generating modality, a picture archiving communication system, a radiology information system, and a laboratory information system.
